SEQUENCE LISTING (45 sequences)

SEQ ID NO: 1 - DNA, 1038 bp, drosophila mauritiana
  misc_feature: Gene sequence encoding the transposase from the Mos-1 transposon
atgtcgagtttcgtgccgaataaagagcaaacgcggacagtattaattttctgttttcatttgaagaaaacagctgcggaatcgcaccgaatgcttgttgaagcctttggcgaacaagtaccaactgtgaaaacgtgtgaacggtggtttcaacgcttcaaaagtggtgattttgacgtcgacgacaaagagcacggaaaaccgccaaaaaggtacgaagacgccgaactgcaagcattattggatgaagacgatgctcaaacgcaaaaacaactcgcagagcagttggaagtaagtcaacaagcagtttccaatcgcttgcgagagatgggaaagattcagaaggtcggtagatgggtgccacatgagttgaacgagaggcagatggagaggcgcaaaaacacatgcgaaattttgctttcacgatacaaaaggaagtcgtttttgcatcgtatcgttactggcgatgaaaaatggatcttttttgttaatcctaaacgtaaaaagtcatacgttgatcctggacaaccgtccacatcgactgctcgaccgaatcgctttggcaagaagacgatgctctgtgtttggtgggatcagagcggtgtcatttactatgagctcttgaaacccggcgaaacggtgaatacggcacgctaccaacaacaattgatcaatttgaaccgtgcgcttcagagaaaacgaccggaatatcaaaaaagacaacacagggtcatttttctccatgacaacgctccatcacatacggcaagagcggttcgcgacacgttggaaacactcaattgggaagtgcttccgcatgcggcttactcaccagacctggccccatccgattaccacctattcgcttcgatgggacacgcactcgctgagcagcgcttcgattcttacgaaagtgtgaaaaaatggctcgatgaatggttcgccgcaaaagacgatgagttctactggcgtggaatccacaaattgcccgagagatgggaaaaatgtgtagctagcgacggcaaatactttgaataa

SEQ ID NO: 2 - protein, 345 aa, drosophila mauritiana
MSSFVPNKEQTRTVLIFCFHLKKTAAESHRMLVEAFGEQVPTVKKCERWFQRFKSGDFDVDDKEHGKPPKRYEDAELQALLDEDDAQTQKQLAEQLEVSQQAVSNRLREMGKIQKVGRWVPHELNERQMERRKNTCEILLSRYKRKSFLHRIVTGDEKWIFFVSPKRKKSYVDPGQPATSTARPNRFGKKTMLCVWWDQSGVIYYELLKRGETVNTARYQQQLINLNRALQRKRPEYQKRQHRVIFLHDNAPSHTARAVRDTLETLNWEVLPHAAYSPDLAPSDYHLFASMGHALAEQRFDSYESVKKWLDEWFAAKDDEFYWRGIHKLPERWEKCVASDGKYLE

SEQ ID NO: 3 - DNA, 36 bp, artificial
  Forward primer F53Y
ggtggtttcaacgctacaaaagtggtgattttgacg

SEQ ID NO: 4 - DNA, 36 bp, artificial
  Reverse primer F53Y
cgtcaaaatcaccacttttgtagcgttgaaaccacc

SEQ ID NO: 5 - DNA, 28 bp, artificial
  Forward primer Q91R
gctcaaacgcaaaaacgactcgcagagc

SEQ ID NO: 6 - DNA, 28 bp, artificial
  Reverse primer Q91R
gctctgcgagtcgtttttgcgtttgagc

SEQ ID NO: 7 - DNA, 27 bp, artificial
  Forward primer L92A
cgcaaaaacaagccgcagagcagttgg

SEQ ID NO: 8 - DNA, 27 bp, artificial
  Reverse primer L92A
ccaactgctctgcggcttgtttttgcg

SEQ ID NO: 9 - DNA, 27 bp, artificial
  Forward primer L92R
cgcaaaaacaacgcgcagagcagttgg

SEQ ID NO: 10 - DNA, 27 bp, artificial
  Reverse primer L92R
ccaactgctctgcgcgttgtttttgcg

SEQ ID NO: 11 - DNA, 31 bp, artificial
  Forward primer Q100N
gcagttggaagtaagtaaccaagcagtttcc

SEQ ID NO: 12 - DNA, 31 bp, artificial
  Reverse primer Q100N
ggaaactgcttggttacttacttccaactgc

SEQ ID NO: 13 - DNA, 31 bp, artificial
  Forward primer Q100R
gcagttggaagtaagtcgacaagcagtttcc

SEQ ID NO: 14 - DNA, 31 bp, artificial
  Reverse primer Q100R
ggaaactgcttgtcgacttacttccaactgc

SEQ ID NO: 15 - DNA, 31 bp, artificial
  Forward primer Q100E
gcagttggaagtaagtgaacaagcagtttcc

SEQ ID NO: 16 - DNA, 31 bp, artificial
  Reverse primer Q100E
ggaaactgcttgttcacttacttccaactgc

SEQ ID NO: 17 - DNA, 44 bp, artificial
  Forward primer S104P
ggaagtaagtcaacaagcagttcccaatcgcttgcgagagatgg

SEQ ID NO: 18 - DNA, 44 bp, artificial
  Reverse primer S104P
ccatctctcgcaagcgattgggaactgcttgttgacttacttcc

SEQ ID NO: 19 - DNA, 25 bp, artificial
  Forward primer N105A
caagcagtttccgcacgcttgcgag

SEQ ID NO: 20 - DNA, 25 bp, artificial
  Reverse primer N105A
ctcgcaagcgtgcggaaactgcttg

SEQ ID NO: 21 - DNA, 34 bp, artificial
  Forward primer E137K
ggcgcaaaaacacatgcaaaattttgctttcacg

SEQ ID NO: 22 - DNA, 34 bp, artificial
  Reverse primer E137K
cgtgaaagcaaaattttgcatgtgtttttgcgcc

SEQ ID NO: 23 - DNA, 27 bp, artificial
  Forward primer T126A
gcgaaacggtgaatgcggcacgctacc

SEQ ID NO: 24 - DNA, 27 bp, artificial
  Reverse primer T126A
ggtagcgtgccgcattcaccgtttcgc

SEQ ID NO: 25 - DNA, 43 bp, artificial
  Forward primer Y237C
gcttcagagaaaacgaccggaatgtcaaaaaagacaacacagg

SEQ ID NO: 26 - DNA, 43 bp, artificial
  Reverse primer Y237C
cctgtgttgtcttttttgacattccggtcgttttctctgaagc

SEQ ID NO: 27 - DNA, 33 bp, artificial
  Forward primer W268F
cgttggaaacactcaatttcgaagtgcttccgc

SEQ ID NO: 28 - DNA, 33 bp, artificial
  Reverse primer W268F
gcggaagcacttcgaaattgagtgtttccaacg

SEQ ID NO: 29 - DNA, 33 bp, artificial
  Forward primer W268Y
cgttggaaacactcaattacgaagtgcttccgc

SEQ ID NO: 30 - DNA, 33 bp, artificial
  Reverse primer W268Y
gcggaagcacttcgtaattgagtgtttccaacg

SEQ ID NO: 31 - DNA, 27 bp, artificial
  Forward primer W268A
cgttggaaacactcaatgcggaagtgc

SEQ ID NO: 32 - DNA, 27 bp, artificial
  Reverse primer W268A
gcacttccgcattgagtgtttccaacg

SEQ ID NO: 33 - DNA, 24 bp, artificial
  PCR primer MTP up
tacgtaatgtcgagtttcgtgccg

SEQ ID NO: 34 - DNA, 24 bp, artificial
  PCR primer 3′HindIII
cccaagcttattcaaagtatttgc

SEQ ID NO: 35 - DNA, 25 bp, artificial
  R primer for the Selex method
caggtcagttcagcggatcctgtcg

SEQ ID NO: 36 - DNA, 25 bp, artificial
  F primer for the Selex method
gaggcgaattcagtgcaactgcagc

SEQ ID NO: 37 - DNA, 28 bp, drosophila mauritiana
  misc_feature: ITR3′ primer
tcaggtgtacaagtatgaaatgtcgttt

SEQ ID NO: 38 - DNA, 28 bp, artificial
  selex46 sequence
tcaggtgtacaagtatgagatgtcgttt

SEQ ID NO: 39 - DNA, 28 bp, artificial
  selex40 sequence
tcaggtgtacaagtatgtaatgtcgtta

SEQ ID NO: 40 - DNA, 28 bp, artificial
  selex49 sequence
gcgggtgtgaaaatatgaaatattcact

SEQ ID NO: 41 - DNA, 28 bp, artificial
  selex1 sequence
gaagagttataactcaagaaaactgaat

SEQ ID NO: 42 - DNA, 25 bp, artificial
  selex69 sequence
cgattacaattatgaaaactttcct

SEQ ID NO: 43 - DNA, 22 bp, artificial
  selex6 sequence
ggagaaatggtagaatagttta

SEQ ID NO: 44 - DNA, 19 bp, artificial
  selex9 sequence
atagtgtttaaagcttact

SEQ ID NO: 45 - DNA, 34 bp, artificial
  selex60 sequence
gtcagtgatgtcaccaacgtatggaatatcgtat

(19) United States
(12) Patent Application Publication
(10) Pub. No.: US 2010/0291682 A1
(43) Pub. Date: Nov. 18, 2010

(54) SYSTEM FOR TRANSPOSING HYPERACTIVE RECOMBINANT DERIVATIVES OF MOS-1 TRANSPOSON

(76) Inventors: Sylvie Marie-Louise Bigot, Montlouis Sur Loire (FR); Yves Philippe Marcel Bigot, Saint Avertin (FR); Florence Bonnin-Rouleux, Joue Les Tours (FR); Stéphanie Odile Rébecca Germon, Nouzilly (FR); Gwenhael Jergot, Landunvez (FR)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/227,271

(22) PCT Filed: May 15, 2007
(86) PCT No.: PCT/FR2007/000823

§ 371 Date: Mar. 11, 2009

(30) Foreign Application Priority Data
May 15, 2006 (FR) 0604285

(51) Int. Cl. C07H 21/04; C12N 15/79; C12N 9/12; C12N 5/10; C12N 1/21; C12N 15/63

(52) U.S. Cl. 435/455; 435/194; 435/325; 435/419; 435/252.3; 435/252.33; 435/320.1; 536/23.1

(57) ABSTRACT

The invention concerns a system for transposing a hyperactive recombinant derivative of Mos-1 transposon, comprising at least the two following partners: a) a Mos-1 pseudo-transposon in which an exogenous nucleotide sequence of interest replaces the nucleotide sequence encoding the original Mos-1 transposase; and b) a Mos-1 tranposase provided in trans in said pseudo-transposon, at least one of said partners being appropriately genetically modified to improve the transposition frequency of said exogenous nucleotide sequence of interest. Additionally to such systems, the invention concerns hyperactive Mos-1 transposons, hyperactive Mos-1 transposases, kits. The invention further concerns the use of one or more of abovementioned means for carrying out sequence transpositions, and more particularly efficient gene transfers.

The present invention relates to the field of molecular biology relating to transposable elements. The invention relates more specifically to the enhancement of the properties of natural transposition systems from mariner mobile genetic elements, for the purposes of the use thereof in biotechnologies.

The present invention relates to a system for transposing a hyperactive recombinant derivative of Mos-1 transposon, comprising at least the two following partners:

a) a Mos-1 pseudo-transposon in which an exogenous nucleotide sequence of interest replaces the nucleotide sequence encoding the original Mos-1 transposase; and

b) a Mos-1 transposase provided in trans in said pseudo-transposon,

at least one of said partners being appropriately genetically modified to improve the transposition frequency of said exogenous nucleotide sequence of interest.

In addition to such systems, the invention relates to hyperactive Mos-1 transposons, hyperactive Mos-1 transposase, kits.

The present invention further relates to the use of one or more of the abovementioned means for carrying out sequence transpositions, and more particularly efficient gene transfers.

Transposable elements (TE) or mobile genetic elements (MGE) are small DNA fragments, capable of moving from one chromosomal site to another (Renault et al., 1997). Said DNA fragments are characterised by reverse repeated sequences (ITR) located at the 5′ and 3′ terminal positions. An enzyme encoded by TEs themselves, transposase, catalyses the transposition process thereof.

TEs have been identified both in prokaryotes and in eukaryotes (see a reference publication in this field: Craig et al., 2002).

TEs are broken down into two classes according to the transposition mechanism thereof. Firstly, class I elements, or retrotransposons, transpose via the reverse transcription of an RNA intermediate. Secondly, class II elements transpose directly from one chromosomal site to another, via a DNA intermediate, according to a “cut and paste” type mechanism.

In prokaryotes, a large number of TEs have been listed to date. They include, for example insertion sequences such as IS1, and transposons, such as Tn5.

In eukaryotes, class II elements comprise two families: P, PiggyBac, hAT, helitron, Harbinger, En/Spm, Mutator, Transib, Pogo and Tc1-mariner.

Mariner mobile genetic elements (or MLE for mariner-like elements) form a major group of class II TEs, belonging to the Tc-1 mariner superfamily (Plasterk et al., 1999).

The ability of TE transposases to mobilise more or less long, homologous or heterologous DNA fragments, comprising sequences of interest, in order to insert same into target nucleic acids, in particular in the chromosome of a host, has been and still is largely used in the field of biotechnologies, particularly in the field of genetic engineering.

Among TEs, MLEs display particularly advantageous properties for use in biotechnology, particularly in genetic engineering and functional genomics. For example, it is possible to cite in a non-limitative manner the following properties:

(i) MLEs are small, easy-to-manipulate transposons.

(ii) The MLE transposition mechanism is simple. In fact, transposase is capable of catalysing, alone, all the steps of MLE transposition. Moreover, it is necessary and sufficient to ensure the mobility of MLEs in the absence of host factors (Lampe et al., 1996).

(iii) MLEs are characterised by a very extensive ubiquity in prokaryotes and eukaryotes. The first MLE, Dmmar1, also referred to as Mos-1, was discovered in Drosphila mauritiana by Jacobson and Hartl (1985). Subsequently, numerous related elements have been identified in genomes particularly belonging to bacteria, protozoa, fungi, plants, invertebrates, cold-blooded vertebrates and mammals.

(iv) The transpositional activity of MLEs is highly specific, and does not induce host genome “resistance” mechanisms, such as methylation interference phenomena [MIP; Jeong et al. (2002); Martienssen and Colot (2001)] or via RNA [RNAi; Ketting et al. (1999); Tabara et al. (1999)]. Transposition events may be controlled by various factors, such as temperature, the presence of certain divalent cations and pH.

In terms of structure, the mariner Mos-1 element is a compact 1286 by element containing a single open reading frame (ORF) which encodes for a 354 amino acid transposase. The transposase consists of an N-terminal domain involved in the bond with the DNA, dimerisation and tetramerisation and a C-terminal domain containing the active site where a DDE(D) pattern coordinates the catalytic metallic ion. The reading frame is flanked by two inverted terminal sequences (ITR) having 28±2 bp. The two regions located between the ITRs and the ORF are not translated and are referred to as UTRs (“Untranslated Terminal Region”. The two ITRs of Mos-1 differ in terms of sequence by four nucleotides, which indicates that the natural configuration of this element is not optimal for transposition. Moreover, this has been verified by experiments demonstrating that pseudo-transposon bordered by two ITR 3′ of Mos-1 transposes 10,000 times better in vivo in bacteria than that bordered by ITR 5′ and 3′ in natural configurations (Augé-Gouillou et al., 2001b).

The potential applications of MLEs in biotechnology, particularly as non-viral genetic recombination tools, are considerable.

Typically, for in vitro insertional mutagenesis applications, the gene of the transposon encoding for transposase is replaced by a “label” DNA. Transposase is provided in trans in protein form. For in vivo or in vitro gene transfer applications, the gene encoding transposase is replaced by the exogenous DNA to be transferred (in this way, a “pseudo-transposon” is obtained). In this case, transposase is supplied in trans via an expression plasmid, a messenger RNA or the protein itself. However, the transfer of an exogenous DNA using current means, i.e. using a mariner pseudo-transposon, is not without its difficulties, particularly due to very limited transgene integration efficiency and specificity.

However, it is essential to have, in each of these applications, efficient transposition systems.

However, the transposition efficiency of natural MLEs, compared to that of other TEs, remains low. In particular, MLEs appear to be less active in eukaryotes than other class II MGEs. Definitively, the practical interest of natural MLEs has remained limited to date, as it is important, for industrialists or researchers, to have efficient transposition tools, so as to reduce the number of procedures, the cost and the time required to conduct the required transpositions. Failing sufficient efficiency of the transposition systems available, the industrialist or the researcher is currently inclined to give priority, whenever possible, to the use of viral gene transfer systems, in spite of the drawbacks involved, to the detriment of recombinant transposition systems constructed using MLE transposons.

Therefore, at the present time, there is a need for a system with (i) makes it possible to transfer genes efficiently; (ii) displays satisfactory safety in terms of immunogenicity for the host; (iii) guarantees the safety of the host and the environment (absence of contaminations, particularly absence of emergence of recombinant viruses); (iv) is easy to produce.

The present invention specifically makes it possible to meet this need by providing a recombination transposition system which makes use of the advantages of the Mos-1 element (ubiquity, transpositional activity, simplicity of production and use, etc.), while remedying the drawbacks associated with the low transposition efficiency of said element in the wild state.

In this way, in order to meet the existing need in a satisfactory way, the inventors took interest in the Mos-1 MLE element of Drosophila Mauritania which is the most characterised member of this family and the only one naturally active. In addition, the Mos-1 element displays the considerable advantage of being active both in eukaryote cells and in bacteria, which renders its significance evident within the scope of the development of an efficient ubiquitous transposition system.

As it emerges from the various embodiments of the present invention described in detail below, the inventors propose several tools which improve the natural Mos-1 transposition system. These tools, which may be used alone or in combination according to the applications envisaged, comprise:

i) hyperactive mutant Mos-1 transposases;

ii) hyperactive recombinant Mos-1 pseudo-transposons.

In this case, a “pseudo-transposon” is defined as a transposon wherein the gene encoding for transposase has been replaced by an exogenous nucleotide sequence. Therefore, a pseudo-transposon comprises ITR and UTR ends but is devoid of transposase activity. As a result, it has lost the ability to transpose, unless it is associated with an external transposase.

In this way, the inventors took an interest in a system for transposing a hyperactive recombinant derivative of Mos-1 transposon, comprising at least the two following partners:

a) a Mos-1 pseudo-transposon in which an exogenous nucleotide sequence of interest replaces the nucleotide sequence encoding the original Mos-1 transposase; and

b) a Mos-1 transposase provided in trans in said pseudo-transposon,

at least one of said partners being appropriately genetically modified to improve the transposition frequency of said exogenous nucleotide sequence of interest by a factor at least equal to 5, preferentially at least equal to 10.

In the proposed recombinant transposition systems, the two partners, i.e. the pseudo-transposon and transposase, may be genetically modified. In this way, either of the partners or both partners may be genetically modified and hyperactive.

A “nucleotide sequence” or a “nucleic acid” according to the invention complies with the usual meaning in the field of biology. These two expressions cover either DNA or RNA, the former possibly being for example genomic, plasmid, recombinant, complementary (cDNA), and the latter, messenger (mRNA), ribosomal (rRNA), transfer (tRNA). Preferentially, the nucleotide sequences and nucleic acids according to the invention are DNA.

The terms and expressions “activity”, “function”, “biological activity” and “biological function” are equivalent and comply with the usual meaning in the technical field of the invention. Within the scope of the invention, the activity in question is the transposition activity.

In this case, “hyperactivity” corresponds to a greater activity to that observed using a natural Mos-1 transposition system comprising:

a) a Mos-1 pseudo-transposon in which an exogenous nucleotide sequence of interest replaces the nucleotide sequence encoding the original Mos-1 transposase; and

b) wild Mos-1 transposase provided in trans in said pseudo-transposon.

Moreover, “hyperactivity” according to the invention refers to a greater transposition activity than that observed using a system for transposing recombinant Mos-1 comprising:

a) a Mos-1 pseudo-transposon in which

(i) an exogenous nucleotide sequence of interest replaces the nucleotide sequence encoding the original Mos-1 transposase; and

(ii) the wild inverted terminal repeat located in 5′ (ITR 5′) has been mutated such that it is a perfect copy of the wild inverted terminal repeat located in 3′ (ITR 3′); and

b) wild Mos-1 transposase provided in trans in said pseudo-transposon.

Therefore, said pseudo-transposon is bordered by two ITR 3′ (“pseudo-transposon 2 ITR 3′” or “pseudo-transposon 3T3”). The presence of ITR 3′ having already been described as making it possible to improve the transposition efficiency with respect to the natural Mos-1 transposon (Augé-Gouillou et al., 2001b), the above recombinant system is therefore used in this case as a reference to determine whether a recombinant transposition system is “hyperactive” according to the present invention. The reference recombinant system described above is hereinafter referred to as the “reference (transposition) system” or “3T3 (transposition) system”.

The “transposition efficiency” is determined according to the transposition frequency. The transposition efficiency is therefore improved if the transposition frequency is increased. Hereinafter, the terms “improvement of (transposition) activity”, “improvement of transposition” or “improvement of (transposition) efficiency” are used interchangeably, all these expressions referring to the “improvement of transposition frequency”, i.e. the increase thereof.

In order to quantify the “hyperactivity”, a “factor” or “hyperactivity factor” is used in this case which is equal to the ratio of the transposition frequencies according to the following formula:

Hyperactivity factor (F)=(transposition frequency observed with a given recombinant transposition system)/(transposition frequency observed with the 3T3 reference transposition system defined above).

In other words, the transposition frequency of an exogenous nucleotide sequence comprised by a Mos-1 pseudo-transposon in the presence of Mos-1 transposase provided in trans is compared to the transposition frequency of said exogenous nucleotide sequence when it is comprised by the reference Mos-1 pseudo-transposon in the presence of the Mos-1 transposase provided in trans (3T3 system). This method for evaluating the transposition activity is one of the most common practices in the field.

The recombinant transposition systems of interest within the scope of the present invention therefore make it possible to improve the transposition by a factor at least equal to 5. Preferentially, the hyperactivity factor is at least equal to 10 and, preferentially, it is at least equal to 15. More preferentially, it is at least equal to 20, 25, 30, 35, 40, 45, 55, 60 and more.

As it emerges from the examples hereinafter, the genetic modification(s) of the pseudo-transposon and/or transposase is/are specifically selected for the ability thereof to induce transposition hyperactivity. The mutations and combinations or mutations which render the transposase and/or pseudo-transposon and/or the transposition system hyperactive are neither random nor predictable. For the purposes of the selection of the suitable mutations, the inventors used a well-known transposition test in the field. This test has already been described in the literature (particularly in Augé-Gouillou et al., 2001b) and belongs to the general knowledge of those skilled in the art who may, if they wish, use any other suitable test at their disposal.

A first aspect of the present invention relates to a system for transposing a hyperactive recombinant derivative of Mos-1 transposon, comprising at least one Mos-1 pseudo-transposon and one Mos-1 transposase provided in trans.

According to a first embodiment, a system for transposing a hyperactive recombinant derivative of Mos-1 transposon comprises the two following partners:

a) a hyperactive Mos-1 pseudo-transposon in which:

i) at least one of the two untranslated terminal repeats (UTR) and/or at least one of the two inverted terminal repeats (ITR) is/are genetically modified; and

ii) an exogenous nucleotide sequence of interest replaces the nucleotide sequence encoding the original Mos-1 transposase;

said pseudo-transposon being selected from the following pseudo-transposons:

α) ITR3′-UTR3′-exogenous nucleotide sequence of interest-UTR3′-ITR3′ (pseudo-transposon 33seq33),

(β) ITR3′-exogenous nucleotide sequence of interest-UTR3′-ITR3′ (pseudo-transposon 3seq33),

γ) ITR3′-UTR5′-exogenous nucleotide sequence of interest-UTR3′-ITR5′ (pseudo-transposon 35seq35),

δ) the pseudo-transposons comprising at least one ITR40 having the sequence SEQ ID No. 39, and

ε) the pseudo-transposons comprising at least one ITR46 having the sequence SEQ ID No. 38;

and

b) a Mos-1 transposase provided in trans in said pseudo-transposon,

the transposition frequency of the exogenous nucleotide sequence of interest of said system being improved by a factor at least equal to 5, preferentially at least equal to 10.

Within the scope of the present invention, the pseudo-transposons are described as follows: ITR-UTR—exogenous nucleotide sequence of interest—UTR-ITR. In the case of the specific example ITR 3′-UTR 3′—exogenous nucleotide sequence of interest—UTR 3′-ITR 3′, this gives the abbreviation 33seq33 or 33T33. The terms 33T33 may more specifically refer to the pseudo-transposon wherein the exogenous nucleotide sequence of interest is the tetracycline resistance gene (see examples below in particular). Naturally, this will clearly emerge from the context.

As specified above, the Mos-1 transposase provided in trans in said system may be a mutant Mos-1 transposase and, in particular, a hyperactive mutant Mos-1 transposase. For example, a suitable hyperactive mutant Mos-1 transposase will comprise at least one mutation on at least one residue selected from the following residues of the sequence SEQ ID No. 2: F53, Q91, E137, T216 and Y237.

In this way, preferred transposition systems according to the present invention comprise at least the two following partners:

a) a hyperactive Mos-1 pseudo-transposon comprising at least one ITR40 having the sequence SEQ ID No. 39 and a hyperactive mutant Mos-1 transposase comprising the mutations T216A and Y237C;

b) a hyperactive Mos-1 pseudo-transposon comprising at least one ITR46 having the sequence SEQ ID No. 38 and a hyperactive mutant Mos-1 transposase comprising the mutations T216A and Y237C, or E137K and T216A, or F53Y and T216A and Y237C;

c) a hyperactive Mos-1 pseudo-transposon 3seq33 and a hyperactive mutant Mos-1 transposase comprising the mutations T216A and Y237C, or E137K and T216A, or F53Y and Q91R, or F53Y and Q91R and E137K and T216A.

According to a second embodiment, a system for transposing a hyperactive recombinant according to the present invention comprises at least the two following partners:

a) a Mos-1 pseudo-transposon in which an exogenous nucleotide sequence of interest replaces the nucleotide sequence encoding the original Mos-1 transposase; and

b) a hyperactive Mos-1 transposase provided in trans in said pseudo-transposon and comprising at least:
one mutation on at least one residue selected from the following residues of the sequence SEQ ID No. 2: F53, Q91 and Y237, and/or
the mutation T216A,

the transposition frequency of the exogenous nucleotide sequence of interest of said system being improved by a factor at least equal to 5, preferentially at least equal to 10.

Preferentially, the hyperactive Mos-1 transposase comprises at least one mutation selected from the mutations F53Y, Q91R, T216A, Y237C, and the combinations thereof. In addition, it may comprise a mutation on the residue E137, particularly the mutation E137K, but excluding the combination of mutations Q91R+E137K+T216A or F53Y+E137K+T216A.

Advantageously, in such a system, at least one of the two untranslated terminal repeats (UTR) and/or at least one of the two inverted terminal repeats (ITR) of the Mos-1 pseudo-transposon may be genetically modified.

In the transposition systems according to the present invention, the wild or genetically modified Mos-1 transposase is provided in trans in the pseudo-transposon. In this way, it may be encoded by a nucleotide sequence placed on a vector, under the control of expression regulation elements. Advantageously, the expression of transposase will then be inducible. For this, conventional promoters may be used by those skilled in the art. For example, they may use the well-known IPTG-inducible promoter pLac. Alternatively, the transposase may be added in the transposition system in the form of a protein or a purified functional protein fraction. Also, it may be supplied in the form of a messenger RNA. In this case, the expression of transposase will be limited in time (to a few hours) due to the lability of messenger RNA.

Particularly advantageously, the exogenous nucleotide sequence of interest comprised by the Mos-1 pseudo-transposon is a functional gene. According to the invention, a gene is said to be “functional” if the corresponding nucleotide sequence comprises at least the open reading frame (ORF), i.e. the encoding sequence, capable of giving rise to an amino acid sequence displaying the activity of the wild gene product. Preferentially, the functional gene is the wild gene. Nevertheless, it may consist of a gene comprising one or more mutations once the product of the mutated gene remains active (even if the activity of the product of the mutated gene is lower than that of the native gene product). In this way, this definition of “functional gene” also covers the encoding sequences devoid of the promoter thereof. For example, the exogenous nucleotide sequence of interest may be a resistance gene to an antibiotic, devoid or not of the promoter thereof (for example, the tetracycline resistance gene), or any other suitable selection marker.

According to the invention, a “mutation” complies with the usual meaning in biotechnology. In this way, a mutation may be a substitution, an addition or deletion of one or more bases in a nucleotide sequence, or of one or more amino acids in a protein sequence. A “mutation” may in particular refer to a substitution of at least one base of a codon of a nucleotide sequence, said substitution inducing for example, during the translation of the nucleotide sequence in question, the incorporation of a different amino acid instead of the native amino acid, in the resulting protein sequence. As a general rule, it will be preferential for the mutation(s) not to induce a loss of the biological function of the mutated product. On the other hand, a decrease in activity may be tolerated, unless the mutated element (for example the pseudo-transposon and/or transposase) is “hyperactive”, in which case the activity thereof should be greater than that of the corresponding wild element.

In this way, a transposase is considered to be “hyperactive”, or a pseudo-transposon is considered to be “hyperactive”, if the transposition efficiency observed when using same is increased. The increase in activity of the element in question (pseudo-transposon or transposase) is determined during the use thereof in a system for transposing a recombinant according to the invention. The hyperactivity factor may thus be determined and, if the level of transpositional activity obtained reaches the threshold defined within the scope of the invention, the element is said to be “hyperactive”.

As a general rule, a “genetic modification” should in this case be understood as being equivalent to one or more mutations. If an encoding sequence is genetically modified, then, typically, it contains one or more mutations. However, these mutations must not induce a loss of the function of the encoded product. On the other hand, in the case, for example, of a genetically modified Mos-1 transposase, preferentially, an increase in the activity thereof (i.e. a hyperactive recombinant transposase) will be required. If a non-encoding sequence is genetically modified (for example, an ITR or a UTR), then either it contains one or more mutations, or the individual sequence per se will not be modified, but the number of repeats of this sequence and/or the order in the chain of sequences and/or orientation of said sequence with respect to the others, is/are modified with respect to the normal configuration (for example, with respect to wild Mos-1 transposon). Examples of genetic modifications of a non-encoding sequence such as an ITR or a UTR particularly include the deletion of all or part of the sequence, the permutation of the sequence with other sequences present in the environment, etc. In sum, a different arrangement of sequences, whether they are encoding or not, falls within the definition “genetic modifications” according to the invention.

According to the above description, it is possible to use, in the systems for transposing a recombinant according to the present invention, a hyperactive Mos-1 pseudo-transposon in which at least one of the two untranslated terminal repeats (UTR) and/or at least one of the two inverted terminal repeats (ITR) of the Mos-1 pseudo-transposon is/are genetically modified.

Preferentially, the pseudo-transposon 2 ITR 3′ is excluded from the Mos-1 transposons liable to be used within the scope of the invention. As specified above, said pseudo-transposon is used as a reference to determine the hyperactivity of the transposition systems according to the invention.

In some cases, at least one of the two untranslated terminal repeats (UTR) of the hyperactive Mos-1 pseudo-transposon is genetically modified. Alternatively or additionally, at least one of the two inverted terminal repeats (ITR) of the hyperactive Mos-1 pseudo-transposon is genetically modified.

According to the above and as illustrated in the examples below, the genetically modified ITR(s) of the hyperactive recombinant Mos-1 pseudo-transposon may be advantageously selected from the ITRSelex sequences referred to as ITR40 (SEQ ID No. 39) and ITR46 (SEQ ID No. 38).

Alternatively, the hyperactive recombinant Mos-1 pseudo-transposon may comprise a combination of ITRs and UTRs particularly selected from the following combinations: ITR 3′+UTR 3′/UTR 3′+ITR 3′ (referenced 33T33 or 33seq33), ITR 3′/UTR 3′+ITR 3′ (referenced 3T33 or 3seq33).

In some embodiments, the Mos-1 transposase provided in trans in the system for transposing a recombinant is a hyperactive transposase comprising at least one mutation on at least one residue selected from the following residues of the sequence SEQ ID No. 2: F53, Q91, E137, T216 and Y237, excluding the combination of mutations Q91R+E137K+T216A or F53Y+E137K+T216A. The results of the experiments conducted by the inventors show that it is not possible to perform any mutation or combination of mutations on the Mos-1 transposase to obtain a hyperactive recombinant transposase. In particular, it emerges (see experimental section below) that the two combinations of mutations excluded in this case result in an abolition of transposition. In said excluded combinations, the mutations may therefore be considered to be antagonistic.

Advantageously, the hyperactive Mos-1 transposase may comprise at least one mutation selected from the mutations F53Y, Q91R, E137K, T216A, Y237C, and the combinations thereof, excluding the combination of mutations Q91R+E137K+T216A or F53Y+E137K+T216A.

Preferentially, the hyperactive Mos-1 transposase may comprise at least one mutation on the T216 residue. More preferentially, it may comprise at least the mutation T216A. Advantageously, such a hyperactive transposase may also comprise at least one mutation selected from the mutations F53Y, Q91R, E137K, Y237C, and the combinations thereof, excluding the combination of mutations Q91R+E137K+T216A or F53Y+E137K+T216A.

As it emerges from the experimental part below, particularly advantageous hyperactive Mos-1 transposases comprise at least one of the following combinations of mutations:
T216A+Y237C; F53Y+T216A+Y237C: hyperactivity factor at least equal to 20;
F53Y+Q91R+E137K+T216A+Y237C: hyperactivity factor at least equal to 30;
F53Y+E137K+T216A+Y237C: hyperactivity factor at least equal to 45.

According to an alternative or additional embodiment, the Mos-1 transposase provided in trans in the systems for transposing a recombinant according to the present invention is a hyperactive transposase comprising at least one mutation on a phosphorylatable residue, said mutation being suppressive of a phosphorylation site in a eukaryote cell (for example, plant, vertebrate or invertebrate cell). Here again, the envisaged mutation(s), suppressive of one or more phosphorylation site(s) in eukaryote cells, are evidently conservative of the protein enzyme function. Suitable hyperactive mutant transposases are described in the French patent application No. 03 00905 filed on 28 Jan. 2003. In particular, said transposases are such that the mutated phosphorylatable residue is selected from the following residues of the sequence SEQ ID No. 2: T11, T24, S28, T42, T88, S99, S104, T135, S147, T154, S170, T181, S200, T216, T255 and S305. Preferentially, the hyperactive transposase comprises at least one mutation on the phosphorylatable residue T88. Advantageously, it also comprises at least one mutation on the phosphorylatable residues T11, T24, S28, T42, S99, S104, T135, S147, T154, S170, T181, S200, T216, T255 and S305. In particular, said phosphorylatable residues mutated in this way are substituted by one or more non-phosphorylatable residues in eukaryote cells.

As a general rule, within the scope of the invention, the hyperactive recombinant or wild Mos-1 transposase may advantageously be produced in a stable manner in prokaryotes. In this case, a suitable process for producing, by a prokaryote host cell, an active (or hyperactive) Mos-1 and stable transposase comprises at least the following steps:

a) the cloning of the nucleotide sequence encoding the active transposase in an expression vector;

b) the cloning of the nucleotide sequence encoding the active catalytic subunit of the cAMP-dependent protein kinase (pKa) in an expression vector;

c) the transformation of said host cell with said expression vectors;

d) the expression of said nucleotide sequences by said host cell; and

e) the obtaining of the active transposase stabilised by phosphorylation by pKa.

Alternatively, the clonings of steps a) and b) are performed in a single expression vector.

Interestingly, such processes also comprise a transposase purification step.

For more details relating to said processes, those skilled in the art may refer to French patent application No. 0512180 filed on 30 Nov. 2005.

A second aspect of the present invention relates to a hyperactive Mos-1 pseudo-transposon, in which:

a) at least one of the two untranslated terminal repeats (UTR) and/or at least one of the two inverted terminal repeats (ITR) is/are genetically modified; and

b) an exogenous nucleotide sequence of interest replaces the nucleotide sequence encoding the original Mos-1 transposase;

said pseudo-transposon being selected from the following pseudo-transposons:

α) ITR3′-UTR3′-exogenous nucleotide sequence of interest-UTR3′-ITR3′ (pseudo-transposon 33seq33)

β) ITR3′-exogenous nucleotide sequence of interest-UTR3′-ITR3′ (pseudo-transposon 3seq33),

γ) ITR3′-UTR5′-exogenous nucleotide sequence of interest-UTR3′-ITR3′ (pseudo-transposon 35seq35),

δ) the pseudo-transposons comprising at least one ITR40 having the sequence SEQ ID No. 39, and

ε) the pseudo-transposons comprising at least one ITR46 having the sequence SEQ ID No. 38.

Such a pseudo-transposon is particularly useful in a system for transposing a hyperactive recombinant as described above.

A third aspect of the present invention relates to a vector comprising at least one pseudo-transposon according to the invention.

In a fourth aspect, the present invention relates to a host cell comprising at least:

a) one system for transposing a recombinant as described above; or

b) one pseudo-transposon according to the invention; or

c) one vector according to the invention; or

d) a combination thereof.

Such a host cell is selected from prokaryote cells (bacteria for example, particularly Escherichia coli) and eukaryote cells (particularly, plant, vertebrate and invertebrate cells).

A fifth aspect of the present invention relates to a kit comprising at least:

a) one transposition system according to the invention; or

b) one pseudo-transposon according to the invention; or

c) one vector according to the invention; or

d) one host cell according to the invention; or

e) a combination thereof.

For example, such a kit may also comprise one or more elements selected from, in particular, a buffer compatible with the transposase(s), a “stop” buffer to stop the transposition reactions, one or more control DNAs (reaction controls), oligonucleotides useful for post-reaction sequencing, competent bacteria, instructions for use, etc.

In a sixth aspect, the present invention relates to uses of at least one of the means described above, i.e. at least:

a) one transposition system; or

b) one pseudo-transposon; or

c) one vector; or

d) one host cell; or

e) one kit; or

f) a combination thereof.

In one embodiment, at least one of these means is used for the efficient in vitro or in vivo (particularly in a plant host cell) or ex vivo transposition of an exogenous nucleotide sequence of interest.

According to another embodiment, at least one of these means is used for the preparation of a medicinal product intended to enable the efficient in vivo transposition of an exogenous nucleotide sequence of interest.

Alternatively, at least one of these means is used for the preparation of a medicinal product resulting from the in vitro or ex vivo transposition of a transposable DNA sequence of interest (exogenous nucleotide sequence of interest) in a target DNA sequence. For example, the invention proposes a method for preparing a medicinal product, comprising at least one transposition step (e.g. in vitro or ex vivo) of a transposable DNA sequence of interest in a target DNA sequence, said transposition being mediated by at least one of the means of the invention. The medicinal product may thus be prepared ex vivo if the transposition is performed in vitro, or in situ if the transposition takes place in vivo.

The means proposed within the scope of the present invention may for example make it possible to modify cells so as to express a medicinal product protein (i.e. a protein of therapeutic or prophylactic interest, for example, insulin, a specific antibody, etc.). Said means may also make it possible to “correct” cells in order to restore a deficient biological function. According to a further embodiment, at least one of these means is used for insertional mutagenesis, or for sequencing and/or cloning nucleic acids.

These applications involve, as a general rule, the use of in vitro or in vivo transposition (particularly in plant host cells), which falls within the scope of the general knowledge of those skilled in the art in the field of the invention (Ausubel et al., 1994; Craig et al., 2002). More specifically with respect to in vivo transposition, the target DNA sequence is typically the genome of the host, which may be an organism, eukaryote (for example a plant host cell) or prokaryote, or a tissue from an organism, or a cell from an organism or a tissue.

In any case, the numerous applications of the means of the invention, for which same prove to be of considerable interest, make use of conventional molecular biology techniques well known to those skilled in the art.

A seventh aspect of the present invention relates to the use of a hyperactive Mos-1 transposase comprising at least:
one mutation on at least one residue selected from the following residues of the sequence SEQ ID No. 2: F53, Q91 and Y237, and/or
the mutation T216A,

to improve, by a factor at least equal to 5, preferentially at least equal to 10, the transposition frequency of an exogenous nucleotide sequence of interest comprised by a Mos-1 pseudo-transposon in which said exogenous nucleotide sequence of interest replaces the nucleotide sequence encoding the original Mos-1 transposase.

The use of such a transposase is particularly carried out in a system for transposing a hyperactive recombinant as described above.

In particular, said hyperactive Mos-1 transposase will comprise at least one mutation selected from the mutations selected from the mutations F53Y, Q91R, T216A, Y237C, and the combinations thereof. In addition, it may comprise a mutation on the residue E137, particularly the mutation E137K, but excluding the combination of mutations Q91R+E137K+T216A or F53Y+E137K+T216A.

In practice, the hyperactive Mos-1 transposase will generally be encoded by a nucleotide sequence placed on a vector, under the control of expression regulation elements. The expression of transposase will thus be advantageously inducible.

According to the above description, the hyperactive Mos-1 transposase preferentially belongs to a hyperactive recombinant transposition system, in which is provided in trans in a Mos-1 pseudo-transposon as described above. In particular, the exogenous nucleotide sequence of interest contained in the Mos-1 pseudo-transposon is a functional gene. Moreover, at least one of the two untranslated terminal repeats (UTR) and/or at least one of the two inverted terminal repeats (ITR) of the Mos-1 pseudo-transposon is/are genetically modified. In practice, it will be advantageous to use a Mos-1 pseudo-transposon comprised by a vector.

The following figures are given for purely illustrative purposes and in no way limit the subject matter of the present invention.

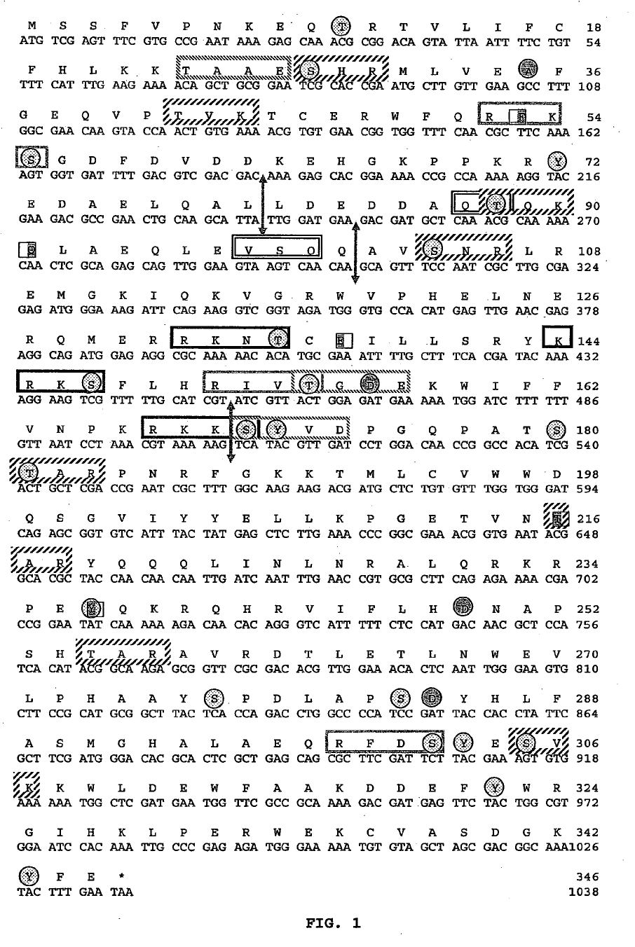

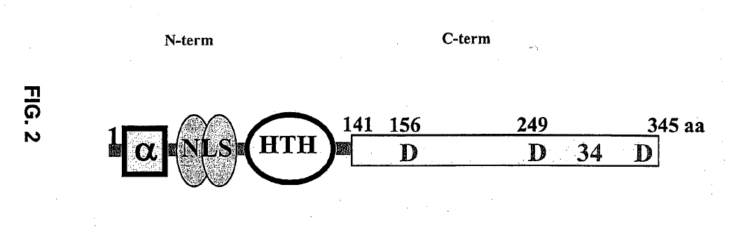

FIG. 1: Nucleotide sequence of the transposase gene of the mos-1 transposon (SEQ ID No. 1) and protein sequence of the Mos-1 transposase (SEQ ID No. 2). The hyperactivity sites (directed mutagenesis target sites) are located by a single border around the residues which appear on a grey background. Putative phosphorylation sites are located as follows:
double border: phosphorylatable amino acid by ATM kinases;
bold hatched border: phosphorylatable amino acid by protein kinase C (pKc);
thick light grey border: phosphorylatable amino acid by cAMP-dependent protein kinase (pKa);
thick black border: phosphorylatable amino acid by pKa and cGMP-dependent protein kinase (pKg);
single hatched border: phosphorylatable amino acid by casein kinase II (CKII).

The residues QTQ (positions 87 to 89 of the protein sequence SEQ ID No. 2) correspond to the putative phosphorylation site by the ATM kinase family. The circles on a dotted background mark highly phosphorylatable residues.

The circles on grey backgrounds identify the residues involved in the characteristic catalytic triad of MLE transposases (D,D34-35[D/E]) and involved in DNA cleavage.

The vertical arrows indicate the proteolytic cleavage sites.

FIG. 2: Diagram representing the structure of the Mos-1 element transposase.

N-term: n-terminal domain responsible for binding with ITRs;

C-term: C-terminal domain responsible for DNA strand transfer catalysis; NLS: putative nuclear localisation (nuclear internationalisation) signal; HTH: helix-turn-helix pattern; aa: amino acid.

The numbers indicate the positions of the amino acids.

The characteristic catalytic triad [D, D34 (D/E)] is signalled.

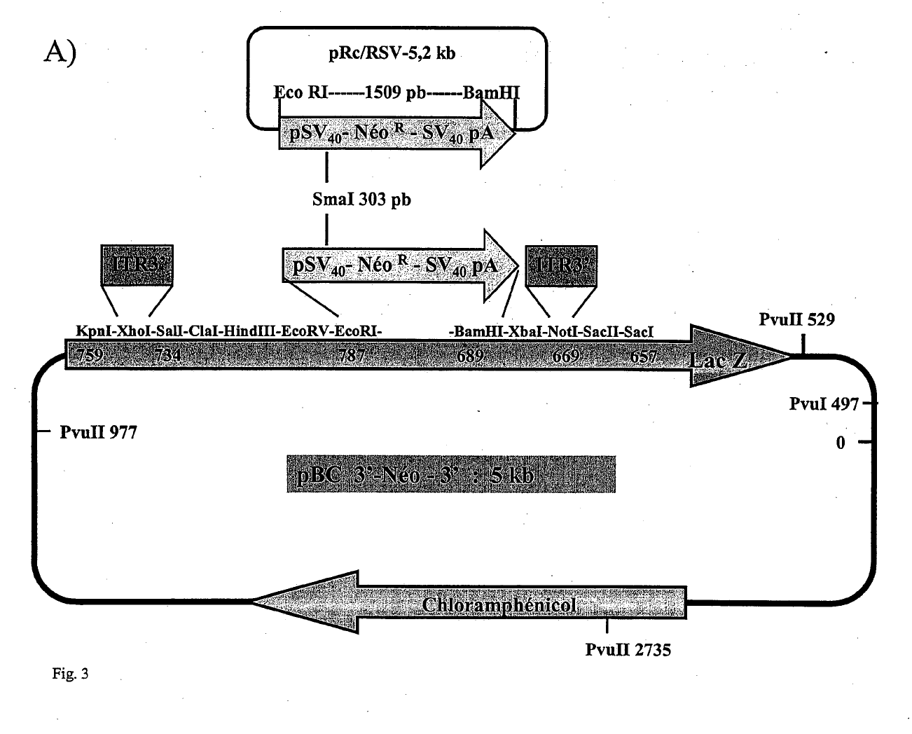

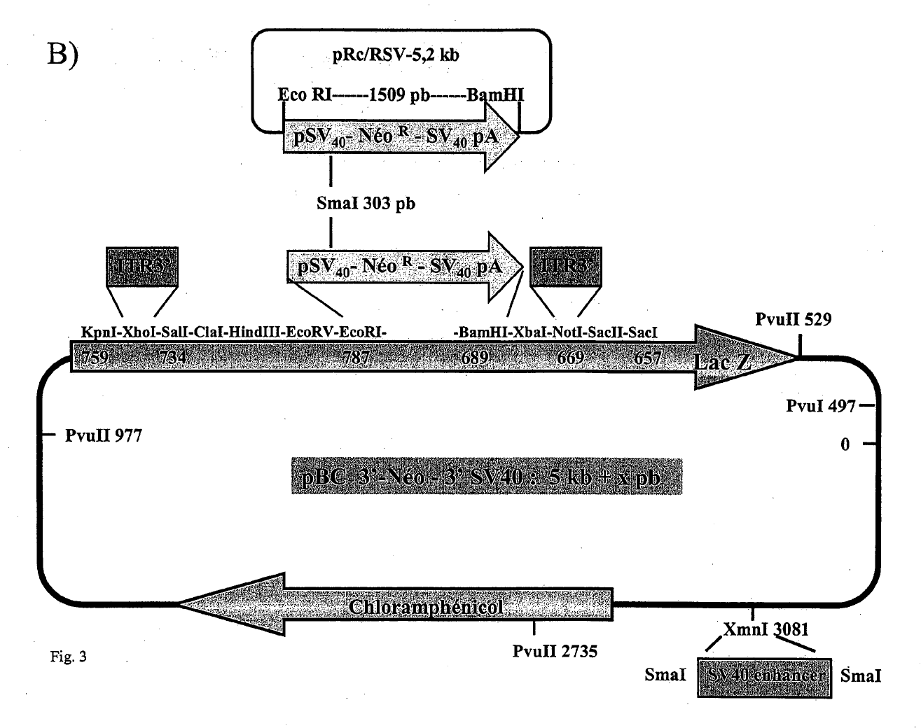

FIG. 3: Schematic representation of pBC3Neo3 plasmid, constructed from pBC SK+ plasmid (Stratagene), and the derivatives thereof (A and B).

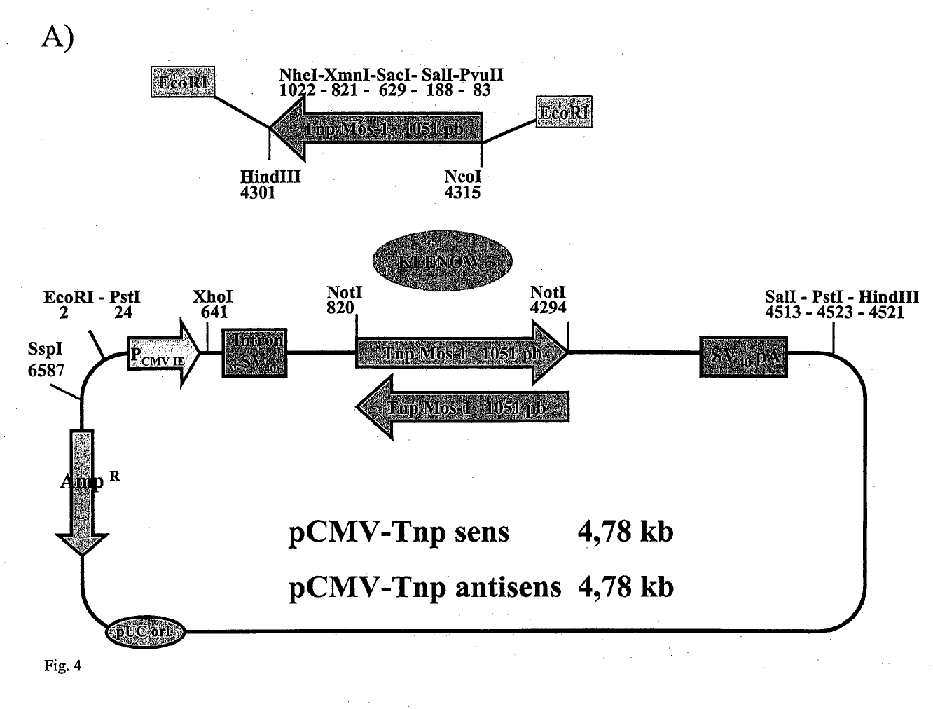

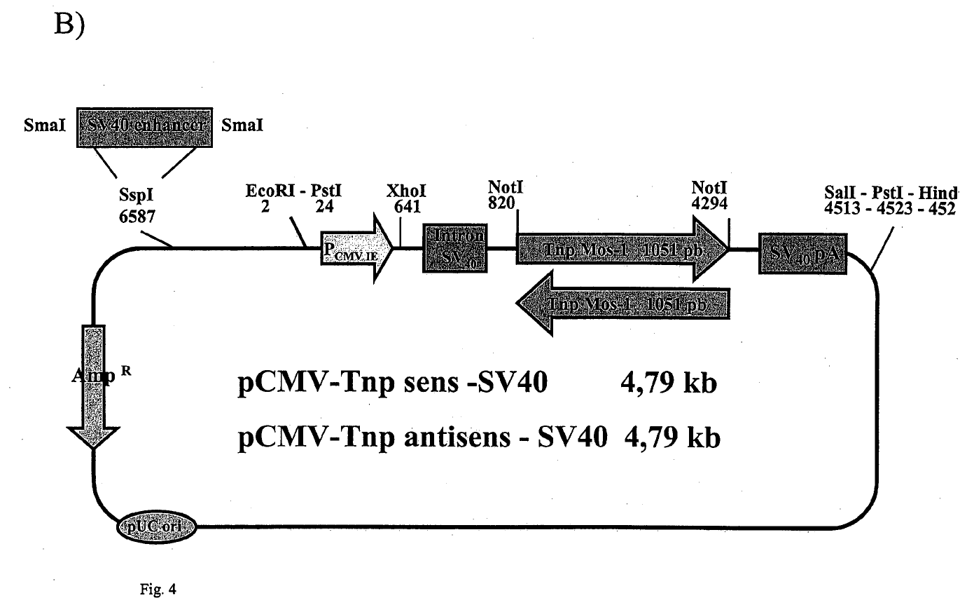

FIG. 4: Schematic representation of pCMV-Tnp plasmid and the derivatives thereof (A and B).

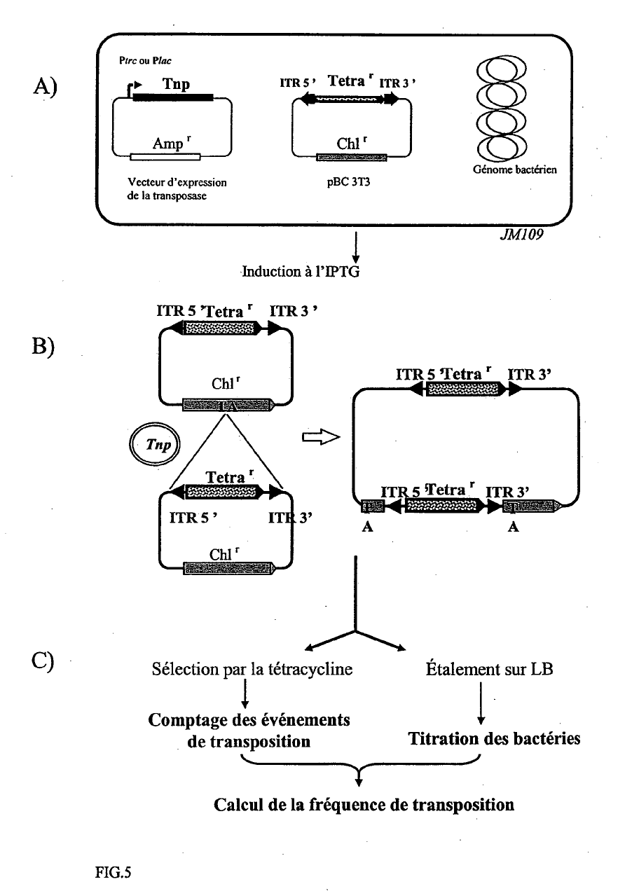

FIG. 5: Schematic representation of transposition test.

A): bacteria co-transformed with the expression vector encoding transposase (Tnp) and the transposition reporter vector.

B): Transposition event after expression vector induction.

C): Determination of transposition frequency.

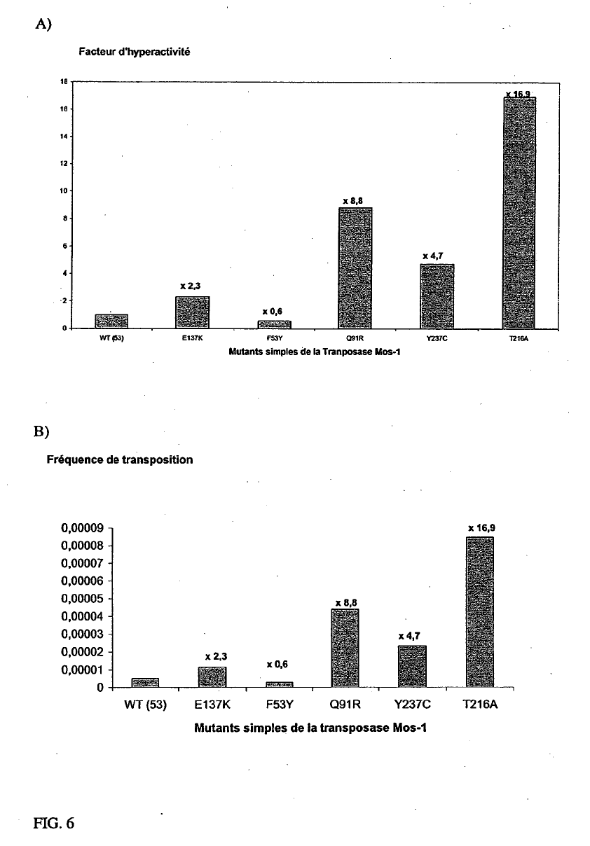

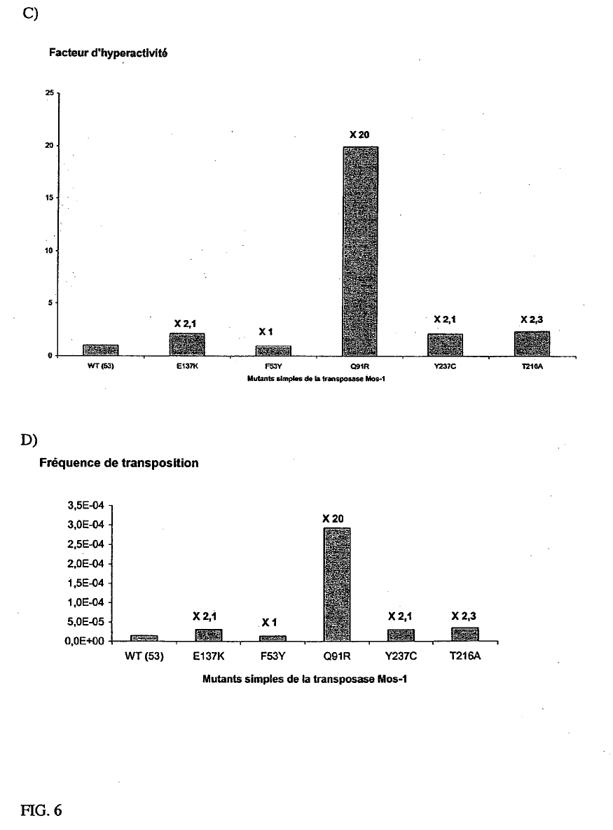

FIG. 6: Mutant transposase transposition efficiency: hyperactivity factor and mutant transposition frequency.

i) Single mutants:
At t=0 hrs after induction: A) Hyperactivity factor; B) Transposition frequency;
At t=5 hrs after induction: C) Hyperactivity factor; D) Transposition frequency;

ii) Multiple mutants:
At t=0 hrs after induction: E) Hyperactivity factor; F) Transposition frequency;
At t=5 hrs after induction: G) Hyperactivity factor; H) Transposition frequency;

WT(53): wild transposase.

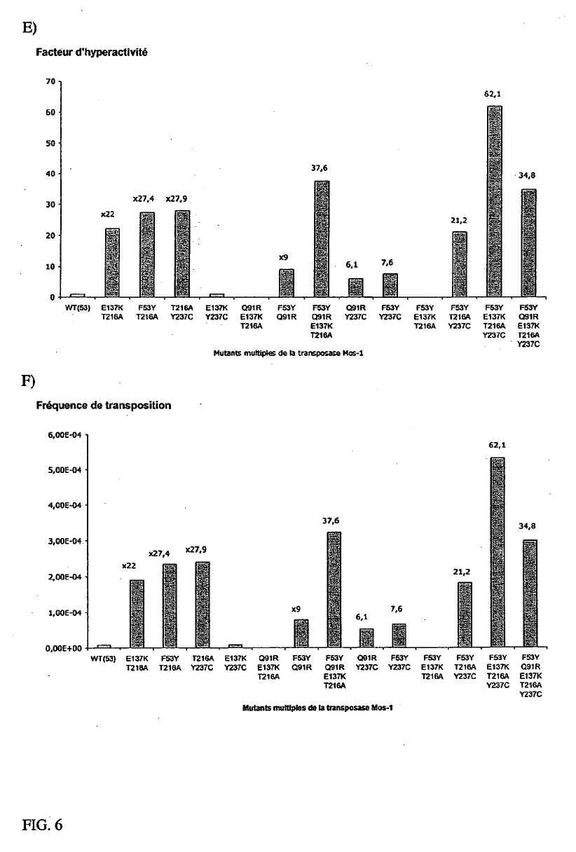

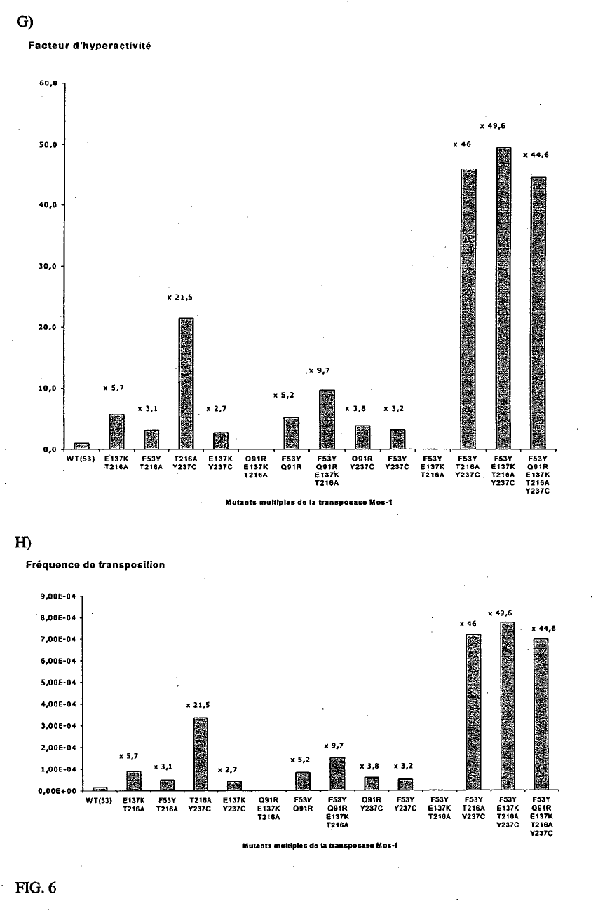

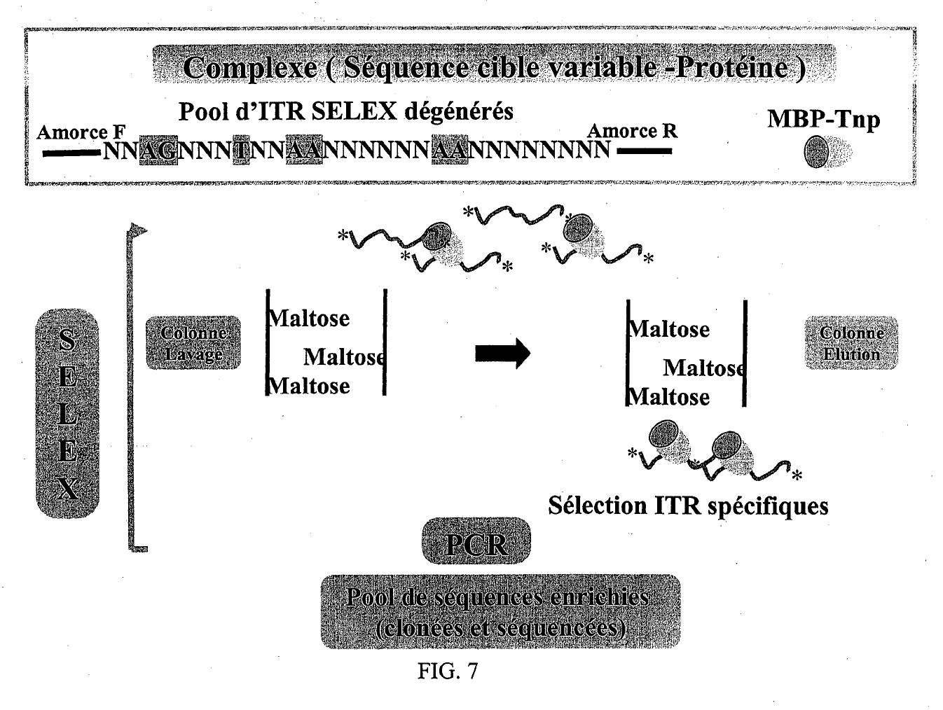

FIG. 7: Diagram illustrating the general principle of the SELEX method.

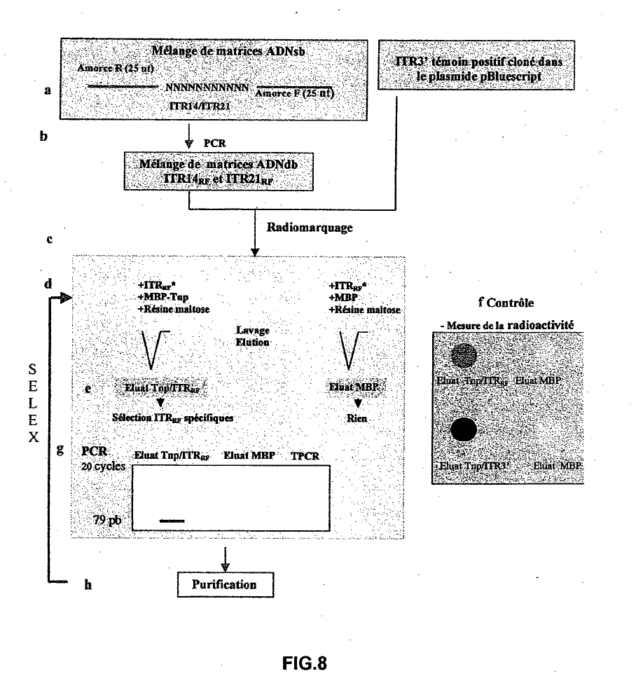

FIG. 8: Diagram illustrating the principle of SELEX method 1.

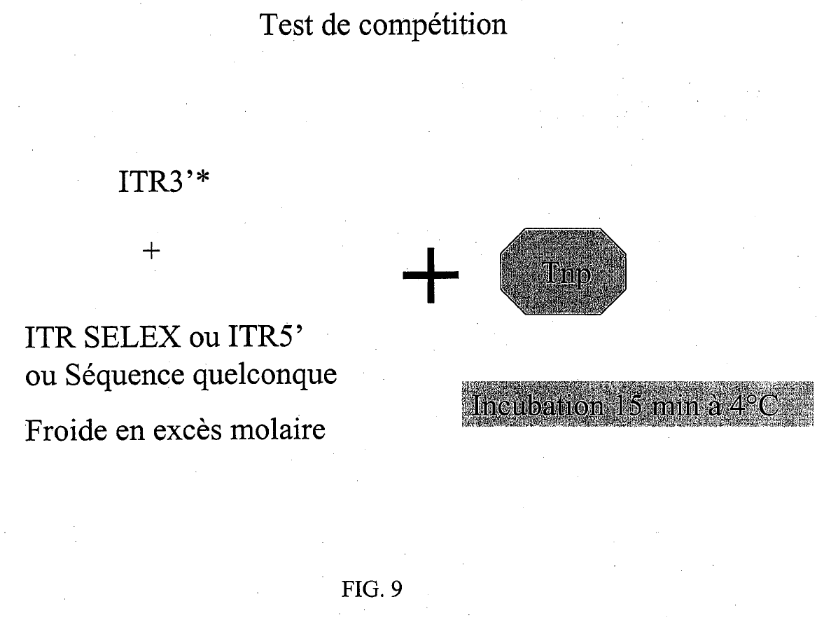

FIG. 9: Diagram representing competition tests.

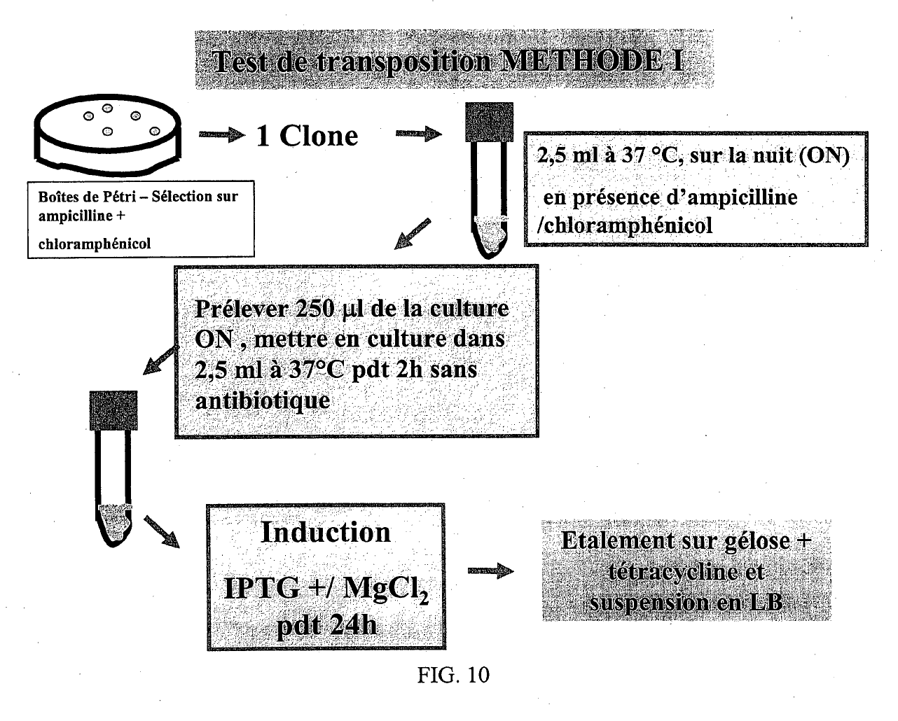

FIG. 10: Diagram representing the operating conditions of in vivo transposition tests in bacteria according to method I.

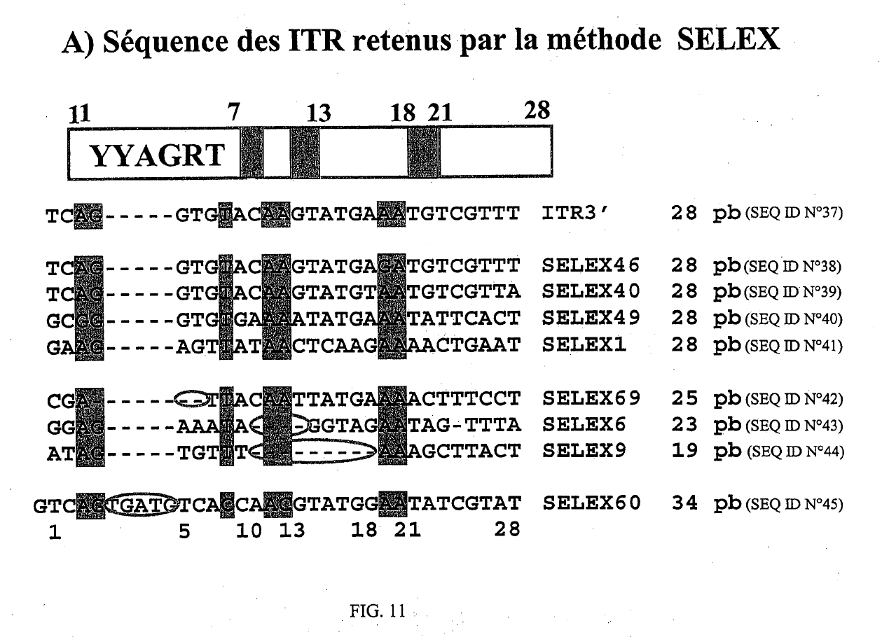

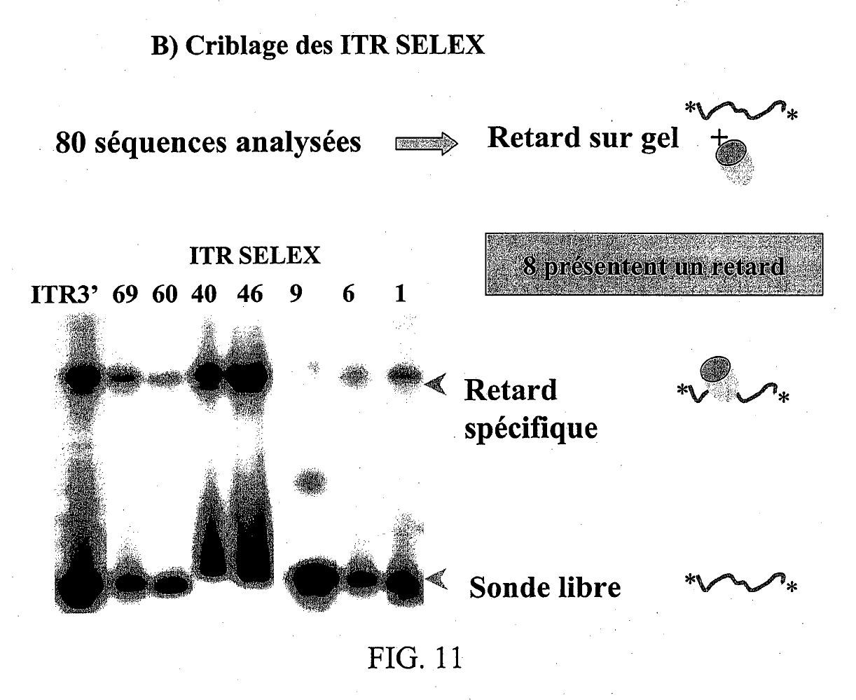

FIG. 11: Results of retardation gels (B) performed with the ITRs present in (A) (ITRSelex selected using SELEX methods developed by the inventors).

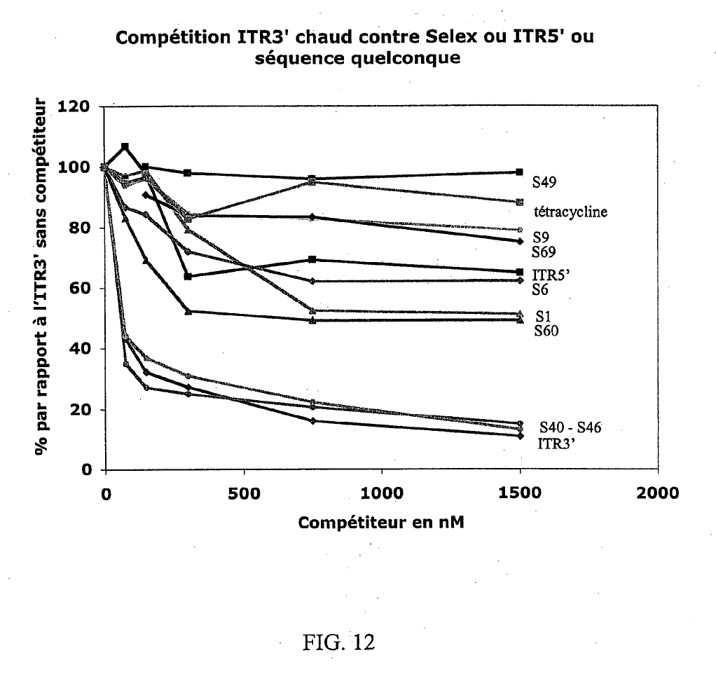

FIG. 12: Results of competition tests.

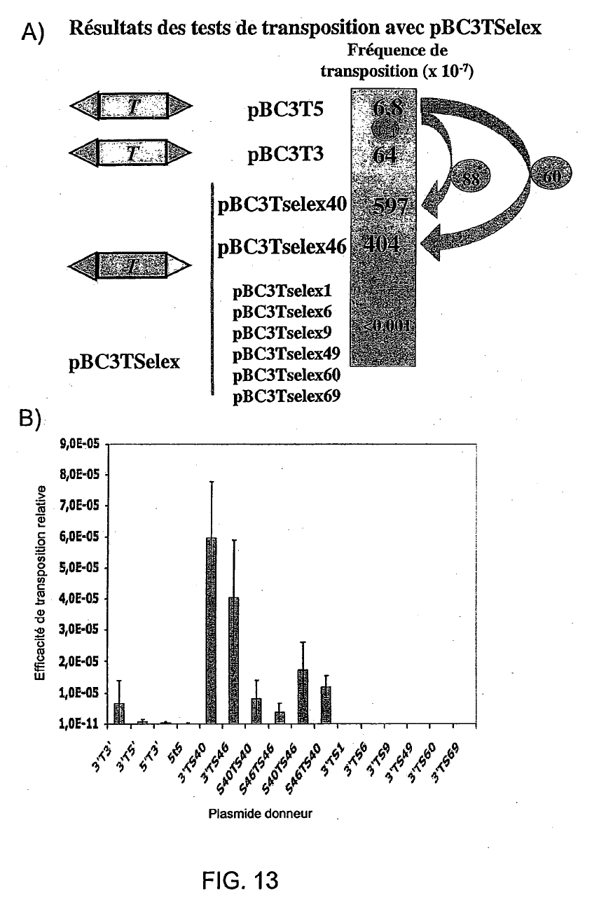

FIG. 13: A) Results of transposition tests obtained with ITRs and B) Supplementary results of transposition tests in bacteria with ITRSelex and wild Mos-1 transposase.

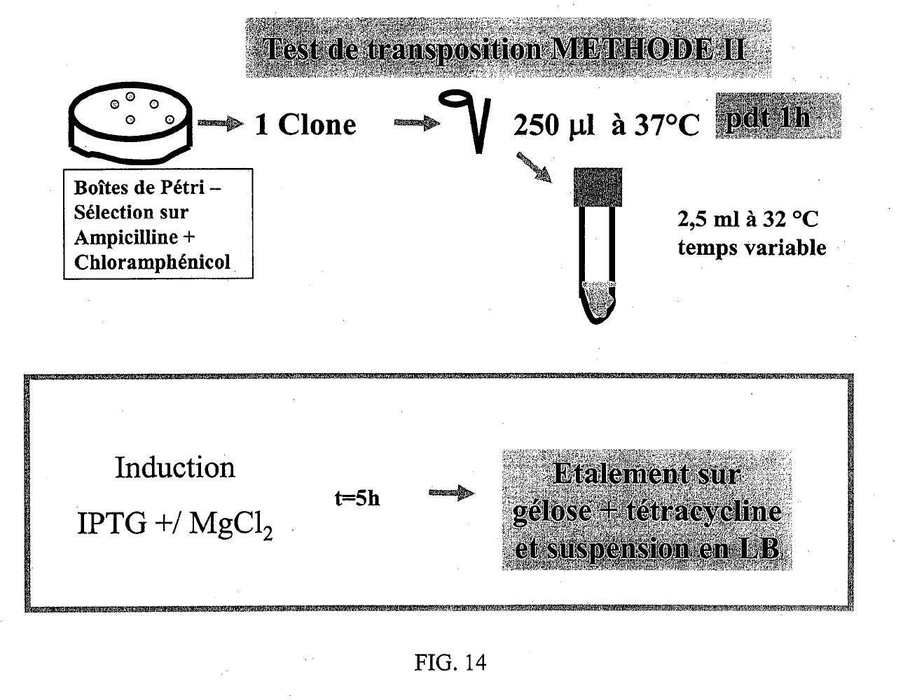

FIG. 14: Diagram illustrating the operating conditions of method II for transposing in vivo in bacteria.

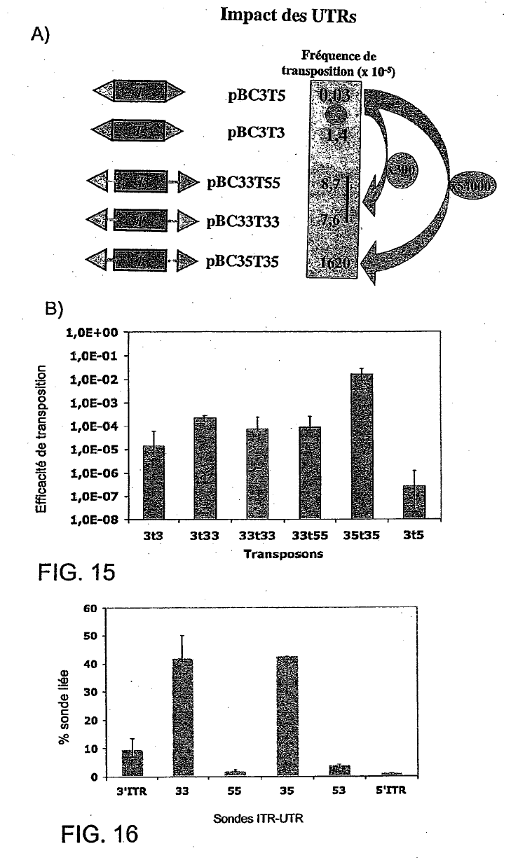

FIG. 15: A) Results of transposition tests obtained with ITRs/UTRs and B) Supplementary results of transposition tests in bacteria with ITR/UTR combinations and wild Mos-1 transposase.

FIG. 16: Graphic representation of quantity of [Wild Mos-1 transposase+ITR] complexes formed with ITR/UTR combinations.

The experimental part below, supported by examples and figures, illustrates the invention in a non-limitative manner.

### EXAMPLES

### Part I

### Hyperactive Mutant Mos-1 Transposases (Tnp)

### I—Materials and Methods

### I-A—Vectors Used

### I-A-1—Description of Plasmids Used

The pGEM-T-Easy vector (3.1 kb) (Promega Charbonnières France; cat. #A1360) comprises the Pu and Prev sequencing primers (Ausubel et al., 1994) and the ampicillin resistance gene. It was devised to clone PCR products in the LacZ gene, which enables white/blue screening of the bacterial colonies obtained on LB ampicillin plates, in the presence of X-Gal and IPTG. It was used to give pGEM-T (Tnp) (Augé-Gouillou et al, 2001). The latter serves as a matrix for transposase mutagenesis before the subcloning thereof in the pKK-233-2 and pCS2+ vectors.

The pKK-Tnp vector (5.6 kb) enables strong expression of the transposase via an IPTG-inducible Plac promoter. The promoter is not modulable and displays natural expression leakage. pKK-Tnp also comprises the ampicillin resistance gene. This plasmid is derived from the pKK-233-2 vector (Clontech, Ozyme, Saint Quentin en Yvelines, France).

The pMalC2x-Tnp vector derived from pMalC2x (New England Biolabs, Ozyme, Saint Quentin en Yvelines, France), enables expression of transposase fused at the N-terminal with MBP protein (Maltose binding protein). It comprises the ampicillin resistance gene.

The pCS2+-Tnp vector is derived from the pCS2+ vector (Turner D L et al., 1994) and enables transposase expression in eukaryote cells under the control of the CMVie promoter. This vector also makes it possible to synthesise in vitro RNA corresponding to the messenger RNA encoding for transposase. Transcription is performed under the control of the SP6 promoter and the RNA is polyadenylated.

pBC 3T3 is a Mos-1 mariner-like donor plasmid (Augé-Gouillou et al, 2001). It contains the “OFF” (i.e. promoter-free) tetracycline resistance gene bordered by two ITR 3′. In this way, only bacteria that have transposed the pseudo-transposon downstream from a promoter (“tagging” promoter) are selected on LB tetracycline plates. The vector comprises the chloramphenicol resistance gene.

pBC3Neo3 (FIG. 3) is a Mos-1 mariner-like donor plasmid. It contains the neomycin resistance gene under the control of the SV40 promoter bordered by two ITR 3′. This enables the selection of eukaryote cells in which the neomycin resistance gene was integrated in the cell genome. The selection is made using G418 (800 μg/ml) for 2 weeks. The vector comprises the chloramphenicol resistance gene.

Therefore, the plasmids pBC 3T3 and pBC3Neo3 contain the Mos-1 pseudo-transposon in which the wild inverted terminal repeat located in 5′ (ITR 5′) has been mutated such that it is a perfect copy of the wild inverted terminal repeat located in 3′ (ITR 3′). Therefore, this pseudo-transposon is bordered by 2 ITR 3′ (“pseudo-transposon 2 ITR3′”). Said pseudo-transposon was used, in association with the wild Mos-1 transposase, to determine the reference transposition frequency, from which the hyperactivity factors associated with the use of the systems according to the invention were determined.

As a general rule, in this type of construction (pseudo-transposon), the letter “T” represents the reporter gene providing tetracycline resistance.

pBC KS Neo is a plasmid containing the neomycin resistance gene under the control of the SV40 promoter. The vector comprises the chloramphenicol resistance gene.

The pGL3-Control vector (Promega; Cat. #E1741) is a plasmid containing the gene encoding for luciferase under the control of the SV40 promoter. It also comprises the ampicillin resistance gene.

### I-A-2—Vector Constructions

### I-A-2-1—Vector DNA Preparation

For the various constructions, all the DNA elutions from an agarose gel were carried out with the Wizard SV Gel and PCR Clean-Up system kit (Promega, France). All the plasmid mini-preparations using bacterial cultures were performed with the Wizard Plus miniprep kit (Promega). Larger-scale DNA preparations were produced with the Pureyield plasmid midiprep system (Promega) or with the Midiprep or Maxiprep kits (Qiagen).

### I-A-2-2—Directed Mutagenesis to Obtain Mutants

The directed mutagenesis was carried out according to the Quikchange site directed mutagenesis kit (Stratagene) protocol. The oligonucleotides used to introduce the mutation were synthesised by MWG Biotech (Roissy CDG). The Pfu polymerase and Dpn1 enzymes were supplied by Promega (France). Briefly, the mutation to be introduced is comprised by two complementary oligonucleotides. The entire plasmid was amplified by PCR (95° C. 1 min followed by 16 cycles 95° C. 30 sec, 55° C. 1 min, 68° C. 2 min/kb of plasmid). The plasmid used as a matrix for the PCR was digested by Dpn1 (1 hr 37° C., 2-3 u/50 μl of PCR). 2-3 μl of the PCR treated with Dpn1 was then transformed in chemocompetent XL1Blue or electrocompetent JM109 bacteria.

The oligonucleotide sequences used for directed mutagenesis are given in Table 1 below and the mutation is specified by the nucleotides in bold type.

TABLE 1

| | | SEQ | | SEQ |
|---|---|---|---|---|
| Muta- | | ID | | ID |
| tion | Forward primer | No. | Reverse primer | No. |
| F53Y | ggtggtttcaacgctaca | 3 | cgtcaaaatcaccactttt | 4 |
| | aaagtggtgattttgacg | | gtagcgttgaaaccacc | |
| Q91R | gctcaaacgcaaaaacga | 5 | gctctgcgagtcgtttttg | 6 |
| | ctcgcagagc | | cgtttgagc | |
| L92A | cgcaaaaacaagccgcag | 7 | ccaactgctctgcggcttg | 8 |
| | agcagttgg | | tttttgcg | |
| L92R | cgcaaaaacaacgcgcag | 9 | ccaactgctctgcgcgttg | 10 |
| | agcagttgg | | tttttgcg | |
| Q100N | gcagttggaagtaagtaa | 11 | ggaaactgcttggttactt | 12 |
| | ccaagcagtttcc | | actttccaactgc | |
| Q100R | gcagttggaagtaagtcg | 13 | ggaaactgcttgtcgactt | 14 |
| | acaagcagtttcc | | actttccaactgc | |
| Q100E | gcagttggaagtaagtga | 15 | ggaaactgcttgttcactt | 16 |
| | acaagcagtttcc | | actttccaactgc | |
| S104P | ggaagtaagtcaacaagc | 17 | ccatctctcgcaagcgatt | 18 |
| | agttcccaatcgcttgcg | | gggaactgcttgttgactt | |
| | agagatgg | | actttcc | |
| N105A | caagcagtttccgcacgc | 19 | ctcgcaagcgtgcggaaac | 20 |
| | ttgcgag | | tgcttg | |
| E137K | ggcgcaaaacacatgcaa | 21 | cgtgaaagcaaaattttgc | 22 |
| | attttgctttcacg | | atgtgtttttgcgcc | |
| T216A | gcgaaacggtgaatgcg | 23 | ggtagcgtgccgcattcac | 24 |
| | gcacgtacc | | cgtttcgc | |
| Y237C | gcttcagagaaaacgac | 25 | cctgtgttgtcttttttga | 26 |
| | cggaatgtcaaaaaaga | | cattccggtcgttttctct | |
| | caacacagg | | gaagc | |
| W268F | cgttggaaacactcaaa | 27 | gcggaagcacttcgaaatt | 28 |
| | tttcgaagtgcttccgc | | gagtgtttccaacg | |
| W268Y | cgttggaaacactcaaa | 29 | gcggaagcacttcgtaatt | 30 |
| | ttacgaagtgcttccgc | | gagtgtttccaacg | |
| W268A | cgttggaaacactcaat | 31 | gcacttccgcattgagtgt | 32 |
| | gcggaagtgc | | ttccaacg | |

### I-A-2-3—Transposase Sequence Verification

The introduction of mutations in the transposase cloned in the pGEM-T easy plasmid was verified by sequencing. For this, 10 microlitres of a DNA mini-preparation were sent for sequencing to MWG Biotech. The primers used (Puniv-21 and Prey-49) were supplied by this company.

I-A-2-4—Sub-Cloning of Mutant Transposases in pKK-233-2 Plasmid.

The fragment encoding for the wild or mutant transposase (Tnp) was prepared from the pGEM-T vector (Tnp) by Nco1/HindIII digestion and eluted on 0.8% agarose gel (TAE1X: 0.04M Tris-Acetate, 1 mM EDTA pH8). The pKK-233-2 plasmid was digested by HindIII/Nco1, deposited on agarose gel, eluted and ligated with the fragment encoding for Mos-1 transposase (referred to as Tnp), overnight at 16° C. A plasmid recircularisation self-check was carried out by performing ligation of the plasmid in the absence of the fragment encoding for transposase.

The ligation product was used to convert E. coli JM109 bacteria which were then selected on LB-ampicillin plates (100 μg/ml). Four ampicillin-resistant clones were placed in culture for plasmid extraction. The DNA mini-preparations were tested by means of EcoR1/HindIII digestion followed by electrophoresis on 0.8% agarose gel (TAE 1×) in order to ensure that they had integrated the gene encoding for transposase.

### I-A-2-5—Sub-Cloning of Mutant Transposases in PMalC2X Plasmid.

For sub-cloning in pMalC2X, the gene encoding for transposase needed to be reamplified by means of PCR using the MTP up and 3′ HindIII primers:

| MTP up: | |
|---|---|
| 5′-TACGTAATGTCGAGTTTCGTGCCG | (SEQ ID No. 33) |
| 3′HindIII: | |
| 5′-CCCAAGCTTATTCAAAGTATTTGC | (SEQ ID No. 34) |

Cycle conditions: 95° C. 5 min followed by 20 cycles (95° C. 30 sec, 50° C. 1 min, 72° C. 1 min) followed by 72° C. 5 min.

The PCR product was then deposited on gel, eluted in 50 μl and cloned in the pGEM-T easy plasmid (1 μl of vector+2 μl of eluted PCR product). After ligation overnight at 16° C., the ligation product was converted into JM109 cells by electroporation and the bacteria were selected on LB Ampicillin plates (100 μg/ml) containing 1 mM IPTG 2% X-Gal. Two ampicillin-resistant blank clones were placed in culture to extract the plasmid DNA. After testing the cloning by means of EcoR1 digestion and electrophoresis on 0.8% agarose gel (TAE 1×), the plasmid was sent for sequencing to MWG

Biotech.

After sequence verification, the fragment encoding for transposase was prepared by means of SnaB1/HindIII digestion and eluted on gel. It was ligated with the pMalC2X plasmid opened by Xmn1/HindIII. JM109 bacteria were then converted by the ligation product and spreaded on LB Ampicillin plates (100 μg/ml).

I-A-2-6—Sub-Cloning of Mutant Transposases in pCS2+ Plasmid

The fragment encoding for the wild or mutant transposase (Tnp) was prepared from the pGEM-T (Tnp) vector by means of EcoR digestion and eluted on 0.8% agarose gel (TAE1X: 0.04M Tris-Acetate, 1 mM EDTA pH8). The pCS2+ plasmid was opened by EcoR1, dephosphorylated and ligated with the fragment containing the transposase. The ligation product was transformed in JM109 bacteria and the clones were selected on LB Ampicillin plates (100 μg/ml). Eight clones were placed in culture to extract the plasmid DNA. The presence of the insert and the orientation thereof was evaluated by means of Pvull/BamH1 or Pvull only digestion and electrophoresis on 0.8% agarose gel in TAE 1×. The clones are designated+sense when the gene is inserted in the sense enabling the mRNA transcription corresponding to the transposase under the control of the SP6 promoter. The clones are designated—sense when the gene is inserted in the opposite sense to transcription under the control of the SP6 promoter.

### I-B—Analysis of Mutant Transposase Activity in Bacteria: Bacteria Transposition Test

Escherichia coli JM109 bacteria were co-transformed with the pBC 3T3 plasmid (comprising a transposition reporter 2 ITR 3′ mariner-like element and the chloramphenicol resistance gene) and with the inducible vector encoding for the transposase (pKK-Tnp or pMal-Tnp) comprising the ampicillin resistance gene. The bacteria selection was performed on ampicillin (100 μg/ml) and chloramphenicol in order to verify the presence of the two plasmids.

The JM109 bacteria containing both the pBC 3T3 plasmid and the pKK-Tnp (or pMalC2X-Tnp) plasmid were placed in culture for 1 hr at 37° C. in 250 μl of LB. This inoculum was then poured into 5 ml of LB supplemented with 1 mM IPTG. The culture was titrated on LB plates (100 μl of a 1/1000 dilution) and on LB-Tet plates (12.5 μg/ml) (250 μl of the non-diluted culture). The culture induced by IPTG was then cultured for 5 hours at 32° C. (optimal temperature of transposase) under stirring (250 rpm). The bacterial suspension was then titrated on LB plates (100 μl of a 1/250,000 dilution) and on LB-tet plates (100 μl of the undiluted bacterial suspension). The plates were placed at 37° C. overnight. The following day, the colonies were counted on LB and LB-Tet plates, and the transposition frequency (equal to the number of tetracycline bacteria over the number of bacterial per 1 ml of undiluted bacterial suspension) was calculated.

### I-C—Analysis of Mutant Transposase Activity in Eukaryote Cells

I-C-1—Cell Transfection

The day prior to transfection, 2.10⁵ HeLa cells were distributed per well of 6-well plates. The following day, the cells were transfected with 3 μg of DNA (750 ng of pGL3-Control, 750 ng of pCS2/Tnp, 1500 ng of pBC3T3) and PEI (1/10 ratio) (Eurogentec). Each condition was tested in duplicate.

Two days after transfection, the transfection efficiency was evaluating by lysing the cells and evaluating the luciferase activity. The cells of the second well were trypsinised and placed in culture in two 10 cm diameter culture plates in the presence of G418 selection agent (800 μg/ml). The culture medium was changed every two days and the selection pressure was maintained for fifteen days. Five clones per condition were isolated and amplified for 15 days under the selection pressure (200 μg/ml of G418).

### I-C-2—Molecular Analysis of Clones

The genomic DNA of the various clones is extracted and subjected to Southern Blot analysis after enzyme restriction. This method is used to analyse the various G418 resistance cassette insertion sites. The sequencing analysis makes it possible to verify the presence of dinucleotide TA duplication which indicates the actual transposition events and evaluate the frequency of random and transposase-dependent recombination events.

### II—Results

To be able to analyse all the steps of Mos-1 Mariner transposition in a simpler system than eukaryote cells, a bacterial study model was developed. The use of this system made it possible to evaluate, at different times, the transposition efficiency of the various mutant transposases.

The results obtained for simple mutations are shown in FIGS. 6A and 6B for a transposition time T0 after induction. The results are expressed as a median increase factor with respect to the wild transposase. The most advantageous mutations are the mutations located on the positions E137, Q91, Y237, T216. The same analysis, but for a transposition time T=5 hrs after induction, gave the results illustrated by FIGS. 6C and 6D.

The multiple mutants were produced by associating the most promising mutations. Seven double, three triple, two quadruple and 1 quintuple mutants were obtained according to Table 2 below.

TABLE 2

| | |
|---|---|
| Double mutants | F53Y + T216A |
| | F53Y + Y237C |
| | F53Y + Q91R |
| | Q91R + Y237C |
| | E137K + T216A |
| | E137K + Y237C |
| | T216A + Y237C |
| Triple mutants | Q91R + E137K + T216A |
| | F53Y + E137K + T216A |
| | F53Y + T216A + Y237C |
| Quadruple mutants | F53Y + E137K + T216A + |
| | Y237C |
| | F53Y + Q91R + T216A + |
| | Y237C |
| Quintuple mutant | F53Y + Q91R + E137K + |
| | T216A + Y237C |

The results of the transposition tests conducted with multiple mutants are recorded in FIG. 6E to 6H.

Some associations (Q91E E137K T216A; F53Y E137K T216A) induce a complete loss of transposition. The other combinations improve transposition, by at least a factor of 6 for the time T0 (FIG. 6E). The most advantageous combinations are the associations F53Y Q91R E137K T216A, F53Y E137K T216A Y237C and the quintuple mutant (FIGS. 6E and 6F).

Similar results were obtained for a transposition time of 5 hours (FIGS. 6G and 6H).

It should be noted that mutations, also described in the literature as having an effect on some properties of Mos-1 transposase, are not for all that advantageous when it is necessary to identify hyperactive mutant Mos-1 transposases. This is the case, for example, of the mutation S104P, represented in Zhang et al. (2001) as modifying the ability of Mos-1 transposase to create protein interactions. Within the scope of their work, the inventors indeed observed that this mutation induced a complete abolition of the transposition activity of Mos-1 transposase during the use of the transposition tests in bacteria (data not shown).

### Part II

### Hyperactive Recombinant Mos-1 Pseudo-Transposons

### I—ITRs (Inverted Terminal Repeats)

Briefly, the detection of optimised ITR sequences was carried out using the SELEX technique which consists of obtaining by means of a combinatorial method, a set of ITRs (FIG. 7). Only some positions known to be essential for the proper functioning of transposase were retained. Therefore, the nature of the other nucleotides varied at random. The different ITRs were selected and enriched for their ability to fix transposase. Among the ITRs selected, only some were capable of delaying transposase in retardation gel (EMSA technique). The transposase associated with modified ITRs was tested in a transposition test in bacteria to evaluate the impact of the change of ITR sequence on transposition efficiency. For some configurations (which affect for example one or two nucleotides with respect to the wild sequence), the transposition efficiency was improved by a significant factor, particularly by a factor at least equal to 5.

### I-1—Materials and Methods

### a) Development of SELEX Method

The use of the SELEX technique enables the inventors to select ITRs displaying a greater affinity for transposase than wild ITRs, in order to improve the performances of the recombinant Mos-1 pseudo-transposon and the transposition system according to the present invention.

The SELEX method, described in 1990 (Ellington et al., 1990; Tuerk et al., 1990), makes it possible to select nucleic acids in mixtures containing more than 10¹⁵ different molecules, according to specific properties, for example the ability to bind with a protein. The general principle of the method consists of incubating a specific target molecule with a mixture of different sequences (RNA, single-strand or double-strand DNA). The fraction capable of binding with the target molecule is isolated from the rest of the nucleic acids by means of a chromatography column, immunoprecipitation or any other suitable purification technique. Subsequently, the enriched fraction is amplified by means of PCR or RT-PCR and used for another selection round. The repetition of selection and amplification cycles makes it possible to enrich the initial mixture with functional oligonucleotides, also referred to as “aptamers”. The greater the increase in the number of selection and amplification cycles, the greater the increase in the quantity of aptamers, until they are dominant in the oligonucleotide population (for a review on the Selex method, see Klug et al., 1994).

Two SELEX methods, described below, are developed by the inventors.

### A1) Source of Transposase

A recombinant protein combining the ITR fixing qualities by transposase (Tnp) and the maltose fixing properties by maltose binding protein (MBP) was used. This recombinant protein, produced in bacteria and called MBP-Tnp, binds with the ITRs via the specific binding domain of transposase located in N-terminal and MBP makes it possible to purify ITR/transposase complexes on a maltose column.

### A2) Nature of Degenerated Sequence ITRs

To carry out SELEX, a mixture of oligonucleotides having 79 bases was synthesised by MWG Biotech. The general structure of these oligonucleotides comprises the sequences of a degenerated ITR having 29 bases, bordered on each of the ends thereof by the sequences of R and F primers each having 25 bases. These primers having the respective sequences 5′-CAGGTCAGTTCAGCGGATCCTGTCG-3′ (SEQ ID No. 35) and 5′-GAGGCGAATTCAGTGCAACTGCAGC-3′ (SEQ ID No. 36) made it possible, in the subsequent steps of the SELEX, to amplify the selected ITR sequences by means of PCR.

Two separate oligonucleotide mixtures were synthesised; one wherein the ITR having 29 bases is degenerated on 14 positions (ITR14), and one wherein the ITR is degenerated on 21 positions (ITR21). The positions retained at 100%, in all the mariner subfamily elements, were maintained in the ITR14 and ITR21, whereas the positions retained at 60/80% were only maintained in the ITR14. The ITR14 were represented by 2.7×10⁸ sequences, the ITR21 by 4.4×10¹² sequences.

In order to validate the method, the Mos-1 ITR3′ was used as the control.

Each of the ITR14 and ITR21 was rendered double-strand by PCR before the first SELEX round given that transposase fixes on double-strand DNA.

### A3) SELEX Method 1 Principle

This method is illustrated in FIG. 8. It uses a pool of single-strand DNA matrices (ss) formed from an ITR having 29 nucleotides (nt) degenerated on 14 or 21 positions and two R and F primers having 25 nucleotides bordering the ends of ITR14 and ITR21 (a). The matrices are rendered double-strand (ds) by PCR (b). They are then labelled radioactively (c) and incubated in solution with the resin and MBP-Tnp fusion protein (referenced Tnp to simplify the figure) or MBP (d). The interaction reaction is carried out for 24 hours at 4° C. After washing the column, the DNA/protein complexes are purified using a maltose solution (e). The eluates retrieved are referred to as Tnp/ITR eluates when the matrices were incubated with MBP-Tnp and MBP eluates when the matrices were incubated with MBP. In order to monitor the selection after each SELEX round, one aliquot of each eluate is deposited on a nylon membrane and counted (f). The matrices selected at each SELEX round are amplified by PCR (g). The amplified products are then tested on agarose gel. The fragments of interest are purified (h) and used for another selection round.

### DNA/Protein Fixing Step:

In order to monitor the ITR selection at each SELEX round, the nucleotide sequences were radiolabelled, either with T4 polynucleotide kinase, or by PCR. The target sequences were selected by incubation in solution of MBP-Tnp, radiolabelled, ITR14 or ITR21, and maltose resin.

In parallel, two control experiments were carried out. A negative control made it possible to ensure the specificity of the DNA/protein interaction, by incubating MBP which does not have a specific affinity for DNA, with the ITR14 or ITR21. The positive control consisted of incubating ITR3′ with MBP-Tnp, on one hand, and MBP, on the other.

### Washing Step and Elution:

After fixing the protein on the target sequence thereof, a washing step was carried out in order to remove all the unbound oligonucleotides without dissociating the complexes. The elution of the retained complexes was carried out by saturating the resin with maltose. Two types of eluates were obtained. The Tnp/ITR eluate was produced by eluting the series of experiments incubating the target oligonucleotides with the MBP-Tnp recombinant protein. The MBP eluate was produced by eluting the column interacting said target ITRs with MBP.

### Selected Sequence Amplification Step:

The amplification of the selected ITRs was performed directly on the Tnp/ITR eluates and the MBP eluate due to the presence of the R and F primers bordering the ITR sequences (SEQ ID No. 35 and 36). If the selection was effective, a specific PCR signal (having 79 bp) needed to be found for the amplification of the matrices contained in the Tnp/ITR eluate, but not for the matrices of the MBP eluate (as MBP has no affinity for ITRs). This positive fragment was eluted from the agarose gels and radiolabelled with T4 polynucleotide kinase. For some PCR cycles, the labelling was performed directly during the amplification step.

This amplimer was then used as a target enriched with Tnp refined sequence in the next SELEX round.

### A4) SELEX Method 2 Principle

Research conducted by the inventors demonstrated that ITR3′ and ITR5′ have the same dissociation constant but the ability thereof to fix transposase is different. The quantity of active protein in the presence of ITR5′ is 10 times lower than that observed in the presence of ITR3′. This indicates that ITR3′ acts as an activator of the ability of transposase to bind an ITR. Therefore, two items of information are contained in an ITR. The first has an effect on protein activation (impact on Bmax), the second modulates the affinity of transposase for ITR (impact on Kd). In order to account for these data, the SELEX method 2 was developed by the inventors. The principle of this method is identical to that of SELEX method 1. However, the DNA matrices were incubated with the protein for five minutes at 4° C. before fixing on the maltose column. This method should thus make it possible to select ITRs having the ability to activate and bind with transposase.

### A5) Protocols

As a general rule, the experimental procedures used by the inventors are based on conventional techniques well known to those skilled in the art (Ausubel et al., 1994; Sambrook and Russel, 2001).

### (i) Matrix Preparation

### Supplementary Strand Synthesis

Transposase only binds on DNA in double-strand form. The synthesis of the supplementary strand of the target oligonucleotides, ITR14 and ITR21, was carried out by means of primer extension. The reaction was performed as for SELEX method 1 and 2.

### DNA Fragment Purification

The PCR products was analysed on 3% Nusieve agarose gel (FMC) in TAE 1× buffer. The DNA fragments were then purified using agarose gel in order to remove any trace of concatemers.

Radioactive labelling of ITR14 and ITR21

The radioactive labelling of the sequences made it possible to monitor the progression of the selection, at each SELEX cycle. The labelling with [γ³² P]ATP (specific activity greater than 4500 Ci/mmol) was performed with T4 phage polynucleotide kinase (PNK), and the labelling with [α³² P]ATP (specific activity greater than 3000 Ci/mmol) was performed by means of PCR, in the presence of the R and F primers (SEQ ID No. 35 and 36).

### (ii) Target Selection by Transposase

### DNA/Protein Fixing Step

The maltose resin (New-England Biolabs) was equilibrated in buffer 1 (20 mM Tris pH9, 50 mM NaCl, 1 mM DTT). The incubation in solution was performed in a final volume of 1 ml of buffer 1, with 200 μl of resin whereto 50 μg of MBP-Tnp or MBP protein; 200 ng of ITR14, ITR21 or ITR3′; 2 μg of ITR 5′ as the competitor; 5 mM of MgCl₂ and 2 μg of salmon sperm DNA were successively added. The interaction reaction of SLEX method 1 was maintained for 24 hours at 4° C. under constant stirring (300 rpm). In SELEX method 2, the DNA matrices and the MBP-Tnp or MBP protein were incubated for 5 minutes at 4° C. before being passed on the maltose column, the interaction reaction being maintained for only 1 hour at 4° C.

### Resin Washing Step and Complex Elution

At the end of incubation, the resin was washed, the protein/ITR complexes eluted. Two successive elutions were carried out in order to retrieve all the complexes.

(iii) Selected Sequence Amplification Step

After each SELEX round, the selected matrices contained in the Tnp/ITR and MBP eluates were amplified before being used for the next SELEX round.

The PCR reaction comprised 15 to 30 cycles, typically 20 cycles (15 cycles in the case of parasitic fragment amplification).

The reaction medium used for the ITR14 and ITR21 amplification contained in a final volume of 50 μl, the matrix: 10 μl of Tnp/ITR eluate or 10 μl of MBP eluate, in the presence of 5 μl of 10× buffer; 200 μM of dNTP; 2.5 mM of MgCl₂; 1 μM of R and F primers and 5 Taq polymerase units. On agarose gel, the amplification using the Tnp/ITR eluates needed to produce a band of 79 by and 300 by for ITR3′ (positive control). The PCR products were purified using agarose gel, and labelled using PNK and used for another SELEX round.

In the fifth SELEX round, the PCR was carried out in the presence of radioactive material for labelling with [α³² P]ATP.

### b) Selected Sequence Cloning and Sequencing

The purified PCR products from round number 7 of SELEX method 1 and round 8 of SELEX method 2 were cloned in the pGEMT-Easy plasmid (pGEMT-Easy Vector system kit, Promega) under the conditions recommended by the supplier. The ligations in the pGEMT-easy were produced with the SELEX method 1 ITR14, SELEX method 2 ITR14, SELEX method 1 ITR21 and SELEX method 2 ITR21 fragments. The ligations were used to transform DH5α competent bacteria. The plasmid DNA of 20 recombinant clones of each of the ligations was analysed by means of single-strand sequencing. An alignment of the sequences was carried out using CLUSTALW software accessible on the site www.infobiogen.fr.

### c) Rapid Screening of Cloned Sequences

Each of the potential ITRs was tested for the ability thereof to fix on transposase by means of gel retardation. The ITRs were thus incubated in the presence MBP-Tnp which should induce a delay in migration if transposase in fixed on ITR. Firstly, a rapid ITR screening was carried out by incubating the radiolabelled DNA by means of PCR, without purification, in the presence of the protein. Secondly, gel retardation was performed in order to eliminate the background noise caused by artefact sequence amplifications.

### C1) Radioactive Labelling of Cloned Sequences

### (i) PCR Labelling

80 ITRs were selected. These ITRs were labelled by PCR, in the presence of [α³² P] ATP and pU and pREV universal primers, using plasmid DNA minipreparations.

The expected size of the amplified fragments was 79 bp.

### (ii) Klenow Fill Labelling

The positive ITRs following the rapid screening were purified on agarose gel after digestion by means of EcoRI enzyme which makes it possible to eliminate the R and F primers. The ITR3′ was purified after pBluescript-ITR3′ plasmid digestion by EcoRI and BamH1 enzymes. Said purified fragments were radiolabelled using [α³² P]ATP using the Klenow site fill technique.

### C2) DNA/Protein Complex Formation

### (i) Rapid Screening

The ITR/protein complexes were formed with the sequences labelled by PCR following the last SELEX reaction cycle, without prior purification, so as to carry out rapid screening. Said sequences contained an ITR bordered by the R and F primers. The interaction reaction contained, in a final volume of 20 μl: 40 μg of MBP-Tnp or MBP protein; 1 μl of the radioactive PCR reaction; 1 μg of salmon sperm DNA; 2 μl of 50% glycerol; 5 mM of MgCl₂ and 0.5 μM of pRev. The free probes were prepared with 1 μl of radioactive PCR, 2 μl of 50% glycerol and 17 μl of buffer. The interaction reactions were maintained for 15 minutes at 4° C. before being analysed on polyacrylamide.

### (ii) Gel Retardation on Purified Probes

The sequences inducing a delay in migration after incubation with Tnp (positive ITRs) were subjected to a further gel retardation with a purified DNA fragment. The ITR/protein complexes were formed in a final volume of 20 μl containing: 40 μg of MBP-Tnp protein, 1 nM of ITR probe, 1 μg of salmon sperm DNA, 2 μl of 50% glycerol, 5 mM of MgCl₂ and 0.5 μM of pRev. The ITRs only were used at a final concentration of 1 nM in a mixture containing 2 μl of 50% glycerol and 17 μl of buffer. The interaction reactions were maintained for 15 minutes at 4° C. before being analysed on polyacrylamide gel.

### d) Competition Tests

The principle of these tests is illustrated in FIG. 9. This test makes it possible to demonstrate the abilities of ITRSelex to shift the transposase fixation of the radiolabelled ITR3′. The greater the shift, the greater the “improvement” of the ITRSelex sequence with respect to ITR3′. In practice, the transposase fixing reaction is carried out in 20 μl containing 10 mM of Tris pH9 buffer, 0.5 mM of DDT, 5 mM MgCl₂, 5% (vol/vol) of glycerol, 1 μg of herring sperm DNA and 100 ng of BSA, in the presence of 15 nM of radiolabelled ITR3′ and non-radiolabelled ITRSelex. The non-radiolabelled ITRSelex concentrations tested were 0 nM, 15 nM, 75 nM, 150 nM, 300 nM, 750 nM, 1500 nM.

e) pBC3TSelex Plasmid Construction

In order to analyse the behaviour of the 8 Selex ITRs in vivo in bacteria, a series of eight plasmids was constructed from the pBC3T5 plasmid. The pBC3T5 plasmid contains the Tet (tetracycline resistance gene) ORF without promoter, bordered by the Mos-1 ITR3′ and 5′. The Tet gene (cloned between the Xba1 and HindIII restriction sites) is in the reverse orientation of the chloramphenicol resistance gene and the gene encoding for LacZ protein. ITR3′ is delimited by the Kpn I restriction site of the pBCKS+ plasmid in 5′ and by the Sal I restriction site in 3′. ITR5′ is delimited by the SacI restriction sites of the pBCKS+ plasmid in 5′ and by the NotI restriction site in 3′. The ITR5′ of the pBC3T5 plasmid was replaced by the ITRSelex after double digestion by NotI and SacI, generating pBC3TSelex plasmids. The ITRSelex were synthesised in the form of single-strand oligonucleotides by MWG Biotech (Germany). The formation of double-strand ITRSelex was carried out by means of hybridisation so as to generate cohesive NotI and SacI half-sites. The cohesive oligonucleotides were designated such that a TA dinucleotide bordering the ITRSelex in 5′ was arranged outside the pseudo-element. Said phosphorylated double-strand oligonucleotides were joined by T4 ligase DNA at the vector directed by the two enzymes, in order to generate the pBC3TSelex plasmids. Said plasmids are hereinafter referred to as pBC3Ts or pBC3TSelex, followed by the ITRSelex number.

### f) Transposition Tests

A detailed description of the experimental protocol is given in part I, paragraph I-B above.

These tests were carried out on JM109 E. coli bacteria co-transformed by 10 ng of transposase donor plasmid (pKK-Tnp or pKK) and 10 ng of pseudo-transposon donor plasmid (pBC3TSelex). These bacteria were selected on a medium containing ampicillin and chloramphenicol. The operating conditions of these tests (“Method I”) are described in FIG. 10.

### I-2—Results

### a) Screening of ITR Candidate Sequences Obtained by SELEX

The SELEX method developed by the inventors uses a mixture of oligonucleotides having 79 by formed from an ITR having 29 by degenerated on 14 or 21 positioned (ITR14 and ITR21), and bordered at the ends thereof by R and F primers having 25 by (SEQ ID No. 35 and 36). It also uses a recombinant protein fusing the Mos1 transposase (reference Tnp) and MBP.

Two SELEX methods were developed. The general principle of these two methods remains the same. In SELEX method 1, the oligonucleotides, protein and maltose resin are incubated at the same time. In SELEX method 2, the matrices are incubated with the protein for 5 minutes at 4° C. before being placed in contact with the maltose resin.

The ITR14 and ITR21 sequences selected at round 7 of SELEX method 1 and round 8 of SELEX method 2 were cloned. For each method, 20 clones corresponding to the ITR14 and 20 clones corresponding to the ITR21 were isolated and sequenced. These 80 sequences were analysed according to the nature thereof, i.e. if it consists of an ITR14 degenerated on 14 positions or an ITR21 degenerated on 21 positions, and the method from which they are obtained (SELEX method 1 or SELEX method 2). The results demonstrated that the method used could have an effect on the type of selection performed (data not shown).

The 80 sequences were tested with gel retardation to test the binding ability thereof with transposase. The results demonstrated that the clones ITR 1, 6, 9, 40, 46, 49, 60 and 69 (ITRSelex; FIG. 11; SEQ ID No. 38 to 45) are capable of forming a complex with the protein. These positive clones were tested with gel retardation in order to determine whether they are recognised by transposase as ITRs or as targets. The results demonstrated that the clones 1, 40, 46, 49 and 69 are ITRs (FIG. 11 and data not shown). The results did not allow any conclusion for clones 6 and 9 (FIG. 11).

### b) Competition Tests

The results demonstrated that, on the 8 ITRs selected on the basis of the above gel retardation experiments, only ITR40 and 46 are capable of inhibiting transposase fixation on radiolabelled ITR3′.

| (SEQ ID No. 39) | |
|---|---|
| ITR40 | 5′-TCAGGTGTACAAGTATGTAATGTCGTTA-3′; |
| (SEQ ID No. 38) | |
| ITR46 | 5′-TCAGGTGTACAAGTATGAGATGTCGTTT-3′. |

As illustrated in FIG. 12, only ITR40 and 46 are capable of competing with ITR3′ and shifting the transposase fixation of radiolabelled ITR3′.

### c) Transposition Tests

Although ITR40 and 46 appear to be the best candidates on the basis of the competition tests, the behaviour of the 8 ITRs was evaluated in transposition tests in vivo in bacteria, in order to verify whether and the extent to which said ITRs have the ability to mediate the entire transposition.

The results obtained are shown in FIGS. 13 A and B. It appears that only ITR40 and 46 actually make it possible to improve transposition under the test conditions. In the case of the references (or controls), under the experimental conditions of method I, the transposition efficiency of pBC3T3 was increased by a factor 10 with respect to that obtained with the pBC3T5 plasmid (FIG. 13A).

Finally, as shown in FIG. 13B, the pseudo-transposons 3T30 and 3T36 are hyperactive.

### II—UTRs (Untranslated Terminal Repeats)

The minimum nucleic acid configuration for optimal Mos-1 element transposition appears to include, in addition to ITRs, at least a part of UTRs. In fact, in vitro, a resistance marker only bordered by Mos-1 ITR 5′ and 3′ does not transpose, while the addition of the 38 first by of UTR 5′ and the 5 first by of UTR 3′ to the sequences of the respective ITRs is sufficient to restore the wild activity (Tosi et al., 2000).

The need for the presence of UTRs in the vicinity of ITRs was evaluated by constructing a number of possible configurations: UTR in 5′ or 3′, UTR 5′ associated with an ITR 3′, or conversely.

In brief, the results obtained demonstrated that the presence of UTRs favours the transposition (in particular, improvement by a factor at least equal to approximately 5).

### II-1—Materials and Methods

### a) ITR-UTR Configuration Construction

a1) Plasmids

The ITR/UTR plasmids were all constructed from the pBC3T5 plasmid using the same operating method. ITR3′ was replaced after double digestion by the KpnI and SalI enzymes. ITR5′ was replaced by double NotI and SacI digestion. The various ITR/URT 33 and 55 sequences were synthesised and cloned in pCR4-TOPO (Invitrogen) by ATG biosynthetics (Germany). The sequence ITR/UTR35-MCS-UTR/ITR35, i.e. ITR3′/UTR5; -Multiple cloning site (MCS)-UTR3′/ITR5′ was synthesised and cloned in pCR-Script AmpSK(+) (Stratagene) by Intelechon (Germany). This sequence was introduced in pBC by double KpnI and SacI digestion. These sequences were designated such that a TA dinucleotide bordering the ITR/UTR in 5′ is arranged outside the pseudo-element. Some fifteen constructions were produced and evaluated in the transposition tests in bacterial according to method 2. The results are shown for the following plasmids with the reference pBC ITR/UTR-T-UTR/ITR: pBC33T33, pBC33T55 and pBC35T35.

The pKKTnp transposase donor plasmid is a derivative of the pKK233-2 plasmid (Clontech; Amp′) wherein, at the Ncol site, the Mos-1 transposase ORF reference Tnp has been cloned. The expression thereof is under the control of the Ptrc IPTG-inducible promoter (however, said promoter comprises a basic transcriptional activity in the absence of inducer; data not shown). Said pKK233-2 plasmid will simply be referenced pKK hereinafter if the transposase ORF is absent.

A2) Transformation of JM109 E. coli Strains for Transposition Tests

The competent JM109 bacteria were co-transformed with a transposon donor plasmid including the plasmids pBC33T3, pBC3T5, pBC3T3, pBC33T55, pBC35T35, pBC3T33, and the pKKTnp Tnp donor plasmid. Control strains were co-transformed with the same transposon donor plasmids and the pKK control plasmid.

### b) Transposition Tests

A detailed description of the experimental protocol is given in part I, paragraph I-B above.

Some fifteen configurations were tested for in vivo transposition in bacteria according to method II, illustrated by FIG. 14.

### II-2—Results

The transposition efficiency was calculated by dividing the number of Tet^{R} clones appearing by the number of bacteria analysed in the presence of the pKK-Tnp transposase donor plasmid, from which the background noise of the experiment obtained in the presence of the pKK control plasmid was removed. The most significant results were obtained for the constructions 3T33, 33T33 and 35T35, in comparison with the control constructions 3T3, 3T5 and 33T55. The transposition efficiency was increased by a factor of 5 and 20 for the pBC33T33 and pBC3T33 constructions, with respect to the control construction bBC3T3. These results demonstrate that the presence of the UTR sequence is extremely important for the transposition reaction as a 300 times superior transposition efficiency is observed with the pBC33T33 construction with respect to that obtained for the bBC3T5 plasmid and for the pBC35T35 plasmid. The best results were obtained for the pBC35T35 construction, which gives an increase in the transposition efficiency by a factor of 54,000 with respect to bBC3T5 and a factor of 1000 with respect to pBC3T3.

According to FIG. 15A, the advantageous constructions of pBC35T35, pBC3T33 and pBC33T33.

FIG. 15B shows that the pseudo-transposons 3T33, 33T33 and 35T35 are hyperactive.

In addition, the inventors tested the constructions 53T35, 53T33, 35T33, 55T35, 53T55, 55T55, 5T35, 5T33, 3T55, 3T53. In this way, they observed that these constructions did not induce hyperactivity; the efficiency thereof was either equivalent to that of 3T5 or 3T3, or lower or zero (data not shown).

### Part III

### Hyperactive Recombinant Transposition Systems Comprising a Hyperactive Mutant Mos-1 Transposase and a Hyperactive Recombinant Mos-1 Pseudo-Transposon

In order to evaluate the transposition efficiency of systems associating a hyperactive transposase and a pseudo-transposon that is also hyperactive, various combinations were tested in the transposition test in bacteria described in part I, paragraph I-B above.

In this test, the pBC3T3 plasmid was replaced by the hyperactive pseudo-transposons pBC3T33, pBC3T40, pBC3T46. The wild pKK-Tnp plasmid was replaced by pKK type vectors each expressing a specific hyperactive mutant transposase.

Table 3 below gives the results obtained by combining hyperactive transposases (FETY, FQETY, FTY, FT, TY, ET, FQ, FQET, QY) and the pseudo-transposons 3T33, 3T40, 3T36, 33T55 (the latter being used as the control)

TABLE 3

Amplification
factor with
respect to
3T3/WT

| | T0 | T5 |
|---|---|---|
| 3T40/ | | |
| FETY | 4 | 0.1 |
| FQETY | 2.5 | 0.8 |
| FTY | 3 | 2.4 |
| FT | 0 | 2 |
| TY | 1.4 | 36.2 |
| ET | 4.1 | 8.9 |
| FQ | 0.7 | 2.5 |
| FQET | 1.8 | 3.7 |
| QY | 0.3 | 1.3 |
| 3T46/ | | |
| FETY | 6.5 | 2 |
| FQETY | 3.6 | 2.3 |
| FTY | 6.7 | 19.4 |
| FT | 0.1 | 3.7 |
| TY | 2.3 | 61.8 |
| ET | 15.6 | 6.7 |
| FQ | 1.4 | 3.3 |
| FQET | 3.3 | 12.3 |
| QY | 1.7 | 1.7 |
| 33T55/ | | |
| FETY | 1.1 | 1.5 |
| FQETY | 0.4 | 1.6 |
| FTY | 2.1 | 4.6 |
| FT | 0 | 0.1 |
| TY | 0 | 8.4 |
| ET | 0 | 7.1 |
| FQ | 0 | 0.1 |
| FQET | 0 | 0.2 |
| QY | 0 | 0 |
| 3T33/ | | |
| FETY | 24.6 | 51.4 |
| FQETY | 22.4 | 59.7 |
| FTY | 44 | 56 |
| FT | 42.9 | 60 |
| TY | 68.4 | 66.5 |
| ET | 37.6 | 218.5 |
| FQ | 4.3 | 109.3 |
| FQET | 36 | 63.5 |
| QY | 5.2 | 28 |

WT: Wild Mos-1 transposase
F: Mos-1 transposase with F53T mutation
E: Mos-1 transposase with E137K mutation
T: Mos-1 transposase with T216A mutation
Y: Mos-1 transposase with Y237C mutation
Q: Mos-1 transposase with Q91R mutation

Surprisingly and unpredictably, the results obtained on the tested combinations show that not all the combinations of hyperactive Mos-1 transposase with a hyperactive Mos-1 pseudo-transposon are necessarily hyperactive.

The results obtained in this way make it possible to select, as advantageous combinations for the purposes of the present invention, the following associations:
pseudo-transposon 3T40+transposase TY,
pseudo-transposon 3T46+transposase TY or ET or FTY
pseudo-transposon 3T33+transposase TY or ET or FQ or FQET.

Under the transposition test conditions in bacteria reported herein, the combination pseudo-transposon 3T33+transposase ET is the most advantageous as it is respectively more efficient than the association pseudo-transposon 3T3+transposase WT (200 times), pseudo-transposon 3T3+transposase FETY (3.5 times) and pseudo-transposon 3T33+transposase WT (10 times).

A biochemical retardation gel analysis was performed according to the procedures described above [in particular, in the internal patent application WO 2004/078981 published on 16 Sep. 2004 and in Augé-Gouillou et al. (2001b)] in order to determine the stability of the transposase+ITR or transposase+ITR/UTR complexes.

This research demonstrated that DNA fragments associating ITR3′ with TUR3′ and ITR3′ with UTR5′ are much more stable (4 times) than those formed with ITRs alone or other combinations (FIG. 16). This greater complex stability may be the source of the hyperactivity observed.

### REFERENCES

Lampe D J. et al. (1996) EMBO J. 15: 5470-5479
Plasterk RHA. et al. (1999) Trends in genetics 15: 326-332
Renault S. et al. (1997) Virologie 1: 133-144
Jacobson and Hartl (1985) Genetics 111: 57-65
Craig et al. (2002) Mobile DNA II. ASM Press. Washington. USA
Jeong et al. (2002) PNAS 99: 1076-1081
Martienssen and Colot (2001) Science 293: 1070-1074
Ketting et al. (1999) Cell 99: 133-141
Tabara et al. (1999) Cell 99: 123-132
Ausubel et al. (1994) In Janssen, K. (Ed) Current Protocols in Molecular Biology. J. Wiley & Sons, Inc. Massachusetts General Hospital, Harvard Medical School
Augé-Gouillou et al. (2001) Mol. Genet. Genomics 265: 58-65
Augé-Gouillou et al. (2001b) Mol. Genet. Genomics 265: 51-57
Turner D L et al. (1994) Genes Dev. 8: 1434-1447
Sambrook and Russel (2001) Molecular Cloning: a laboratory manual (3^{rd} Ed.) Cold Spring Harbor Laboratory Press, Cold Spring Harbor, N. Y.
Tosi et al. (2000) Nucleic Acids Res. 28: 784-790
Ellington et al. (1990) Nature 346: 818-822
Tuerk et al. (1990) Science 249: 505-510
Klug et al. (1994) Mol. Biol. Rep. 20: 97-107
Zhang et al. (2001) Nucleic Acids Res. 29: 3566-3575

1. A hyperactive Mos-1 pseudo-transposon comprising
a) at least one untranslated terminal repeat (UTR) and/or at least one inverted terminal repeats. (ITR) wherein said UTR and/or said ITR is genetically modified; and
b) an exogenous nucleotide sequence of interest replacing a nucleotide sequence encoding original Mos-1 transposase; and wherein said hyperactive Mos-1 pseudo-transposon
is selected from the group consisting of the following pseudo-transposons:
α) ITR3′-UTR3′-exogenous nucleotide sequence of interest-UTR3′-ITR3′ (pseudo-transposon 33seq33),
β) ITR3′-exogenous nucleotide sequence of interest-UTR3′-ITR3′ (pseudo-transposon 3seq33),
γ) ITR3′-UTR5′-exogenous nucleotide sequence of interest-UTR3′-ITR5′ (pseudo-transposon 35seq35),
δ) pseudo-transposons comprising at least one ITR40 having sequence SEQ ID NO:39, and
ε) pseudo-transposons comprising at least one ITR46 having sequence SEQ ID NO:38.

2. A system for transposing a hyperactive recombinant derivative of Mos-1 transposon comprising
a) the hyperactive Mos-1 pseudo-transposon according to claim 1; and
b) a Mos-1 transposase provided in trans in said hyperactive Mos-1 pseudo-transposon, and
wherein transposition frequency of said exogenous nucleotide sequence of interest is improved by a factor at least equal to 5.

3. The system according to claim 2 wherein said Mos-1 transposase provided in trans is a mutant transposase.

4. The system according to claim 3, wherein said mutant Mos-1 transposase is hyperactive.

5. The system according to claim 4, wherein said hyperactive mutant Mos-1 transposase comprises at least one mutation on at least one residue selected from the group consisting of residues F53, Q91, E137, T216 and Y237 of SEQ ID NO:2.

6. The system according to claim 5, wherein said system comprises
a) a hyperactive Mos-1 pseudo-transposon comprising at least one ITR40 having sequence SEQ ID NO:39 and a hyperactive mutant Mos-1 transposase comprising mutations T216A and Y237C;
b) a hyperactive Mos-1 pseudo-transposon comprising at least one ITR46 having sequence SEQ ID NO:38 and a hyperactive mutant Mos-1 transposase comprising mutations T216A and Y237C, or E137K and T216A, or F53Y and T216A and Y237C; and
c) a hyperactive Mos-1 pseudo-transposon 3seq33 and a hyperactive mutant Mos-1 transposase comprising mutations T216A and Y237C, or E137K and T216A, or F53Y and Q91R, or F53Y and Q91R and E137K and T216A.

7. A system for transposing a hyperactive recombinant derivative of Mos-1 transposon, comprising
a) a Mos-1 pseudo-transposon in which an exogenous nucleotide sequence of interest replaces a nucleotide sequence encoding original Mos-1 transposase; and
b) a hyperactive Mos-1 transposase provided in trans in said Mos-1 pseudo-transposon and comprising at least:
one mutation on at least one residue selected from the group consisting of residues F53, Q91 and Y237 of SEQ ID NO:2, and/or
mutation T216A,
and wherein transposition frequency of said exogenous nucleotide sequence of interest is improved by a factor at least equal to 5.

8. The system according to claim 7, wherein said hyperactive Mos-1 transposase comprises at least one mutation selected from the group consisting of mutations F53Y, Q91R, T216A, and Y237C.

9. The system according to claim 7 wherein said hyperactive Mos-1 transposase also comprises a mutation on residue E137.

10. The system according to claim 9, wherein said hyperactive Mos-1 transposase also comprises mutation E137K, excluding mutations Q91R+E137K+T216A or F53Y+E137K+T216A.

11. The system according to claim 7 wherein at least one untranslated terminal repeat (UTR) and/or at least one inverted terminal repeat (ITR) of the Mos-1 pseudo-transposon is genetically modified.

12. The system according to claim 2 wherein said Mos-1 transposase provided in trans is encoded by a nucleotide sequence placed on a vector, under the control of expression regulation elements.

13. The system according to claim 12 wherein expression of said transposase is inducible.

14. The pseudo-transposon according to claim 1 wherein said exogenous nucleotide sequence of interest is a functional gene.

15. A vector comprising one pseudo-transposon according to claim 1.

16. A host cell comprising
the pseudo-transposon according to claim 1.

17. A kit comprising
the pseudo transposon according to claim 1.

18.-20. (canceled)

21. A method for preparing a medicinal product, wherein said method comprises at least one step of in vitro or ex vivo transposition of a transposable DNA sequence of interest in a target DNA sequence, said transposition being mediated by at least
the pseudo-transposon according to claim 1.

22.-30. (canceled)

31. The system according to claim 2, wherein said transposition frequency is at least equal to 10.

32. The system according to claim 7, wherein said transposition frequency is at least equal to 10.

33. The system according to claim 2 wherein said exogenous nucleotide sequence of interest is a functional gene.

34. A host cell comprising the system according to claim 2.

35. A host cell comprising the system according to claim 7.

36. A host cell comprising the vector according to claim 15.

37. A kit comprising the system according to claim 2.

38. A kit comprising the system according to claim 7.

39. A kit comprising the vector according to claim 15.

40. A kit comprising the host cell according to claim 16.

41. A method for preparing a medicinal product, wherein said method comprises at least one step of in vitro or ex vivo transposition of a transposable DNA sequence of interest in a target DNA sequence, said transposition being mediated by at least the system according to claim 2.

42. A method for preparing a medicinal product, wherein said method comprises at least one step of in vitro or ex vivo transposition of a transposable DNA sequence of interest in a target DNA sequence, said transposition being mediated by at least the system according to claim 7.

43. A method for preparing a medicinal product, wherein said method comprises at least one step of in vitro or ex vivo transposition of a transposable DNA sequence of interest in a target DNA sequence, said transposition being mediated by at least the vector according to claim 15.

44. A method for preparing a medicinal product, wherein said method comprises at least one step of in vitro or ex vivo transposition of a transposable DNA sequence of interest in a target DNA sequence, said transposition being mediated by at least the host cell according to claim 16.

45. A method for preparing a medicinal product, wherein said method comprises at least one step of in vitro or ex vivo transposition of a transposable DNA sequence of interest in a target DNA sequence, said transposition being mediated by at least the kit according to claim 17.
